(19) United States
(12) Patent Application Publication
(10) Pub. No.: US 2022/0151916 A1
(43) Pub. Date: May 19, 2022

(54) SYSTEMS AND METHODS FOR BISTABLE OLIGOMERIC MACHINES

(71) Applicants: Vladik AVETISOV, Moscow (RU); Roman ILIEV, Moscow (RU)

(72) Inventors: Vladik AVETISOV, Moscow (RU); Roman ILIEV, Moscow (RU)

(21) Appl. No.: 17/598,545

(22) PCT Filed: May 27, 2020
(86) PCT No.: PCT/IB2020/000411

§ 371 Date: Sep. 27, 2021

Related U.S. Application Data
(60) Provisional application No. 62/824,610, filed on Mar. 27, 2019.

(51) Int. Cl. A61K 9/00; C08F 293/00; A61K 47/32

(52) U.S. Cl. CPC A61K 9/0009; A61K 47/32; C08F 293/00

(57) ABSTRACT

In some embodiments, molecular and/or oligomeric machines comprising oligomeric modules are selected and joined so as to exhibit conformational bistability wherein a relative orientation between oligomeric modules may change from a first orientation to a second orientation in response to one or more stimuli.

### CROSS REFERENCES TO RELATED APPLICATIONS

This application claims the benefit of priority of U.S. Provisional Patent Application No. 62,824,610, filed on Mar. 27, 2019 the contents of which are incorporated herein by reference in their entirety.

### FIELD

This application is directed towards nanomechanical devices whose functioning is related to conformational bistability of nanoscale oligomeric structures and/or their nanoscale compositions.

### BACKGROUND

Industrial miniaturization of devices and machines is typically carried out by top-down design. The creation of smaller and smaller components and devices is desired, and manufacturing is moving to the nanometer scale from the micrometer scale. Approaching the size of about 10 nm by top-down design, the cost of precise manipulations using macroscopic devices typically increases and may become prohibitively expensive. Alternatively, bottom-up strategies which design functional devices on the nanometer scale from building elements of sub-nanometer (atomic) size may prove beneficial.

Oligomeric machines exhibiting conformational bistability may provide nano-mechanic functionality, afford stimuli responsive control, and enable nanoscale manipulations for various applications including but not limited to energy harvesting, stimuli-responsive mechanical activation, sensing, drug delivery, and biotherapeutics.

### SUMMARY

In some embodiments, molecular and/or oligomeric machines comprising oligomeric modules are selected and joined so as to exhibit conformational bistability wherein a relative orientation between oligomeric modules may change from a first orientation to a second orientation in response to one or more stimuli.

In some embodiments, an oligomeric machine comprises a synthetic material including a first oligomeric module and a second oligomeric module joined to the first oligomeric module to form an oligomeric chain, at least one bending or hinge location at a position of co-joinder between the first oligomeric module and the second oligomeric module, the bending or hinge location permitting relative flexure between the first oligomeric module and the second oligomeric module, at least one electric generating element, a substrate configured relative to the at least one electric generating element and the oligomeric chain such that the relative flexure between the first oligomeric module and the second oligomeric module results in mechanical interaction between at least the second oligomeric module of the oligomeric chain and the at least one electric generating element, and wherein the oligomeric chain is formed such that in response to a stimulus, the relative flexure occurs between the first oligomeric module and the second oligomeric module in a manner causing the mechanical interaction between the second oligomeric module and the electric generating element, and wherein the mechanical interaction produces a change in electrical voltage associated with the at least one electric generating element.

In some embodiments, an oligomeric drug delivery machine comprises a first oligomeric module, a second oligomeric module connected to the first oligomeric module at a bend or hinge location to form an oligomeric chain, and a therapeutic agent captured between the first oligomeric module and the second oligomeric module wherein the oligomeric chain is configured such that upon application of energy thereto, relative movement occurs between the first oligomeric module and the second oligomeric module such that the captured therapeutic agent is released.

In some embodiments, an oligomeric machine comprises an arrangement of molecules configured for introduction into a mammalian body, the molecules being arranged and selected such that when exposed to a prescribed temperature, the arrangement performs at least one mechanical function selected from a group consisting of vibrations, extending, rotating, lifting, pressing, ratcheting, springing, and flexing wherein the prescribed temperature is a normal mammalian body temperature and is below a temperature causing necrosis to mammalian cells, such that upon introduction of the molecular arrangement to the mammalian body, the molecular arrangement does not perform the mechanical function until the arrangement of molecules is exposed to a temperature at least equal to the prescribed temperature.

In some embodiments, an oligomeric machine comprises a synthetic material including a first oligomeric module and a second oligomeric module joined to the first oligomeric module to form an oligomeric chain, at least one bend or hinge location at a position of co-joinder between the first oligomeric module and the second oligomeric module, the bend or hinge location permitting relative flexure between the first oligomeric module and the second oligomeric module, a first chemical reagent attached to the first oligomeric module, a second chemical reagent attached to the second oligomeric module, and wherein the oligomeric chain is formed such that in response to a prescribed amount of energy applied thereto, the first chemical reagent and the second chemical reagent are caused to be drawn into contact with each other and to undergo a chemical reaction.

In some embodiments, an oligomeric machine comprises a synthetic material including a first oligomeric module and a second oligomeric module joined to the first oligomeric module to form an oligomeric chain, at least one bending or hinge location at a position of co-joinder between the first oligomeric module and the second oligomeric module, the bending or hinge location permitting relative flexure between the first oligomeric module and the second oligomeric module, at least one piston element, a substrate configured relative to the at least one piston element and the oligomeric chain such that the relative flexure between the first oligomeric module and the second oligomeric module results in mechanical interaction between at least the second oligomeric module of the oligomeric chain and the at least one piston element, and wherein the oligomeric chain is formed such that in response to a prescribed amount of energy applied thereto, the relative flexure occurs between the first oligomeric module and the second oligomeric module in a manner causing the mechanical interaction between the second oligomeric module and the piston element, and wherein the mechanical interaction produces a mechanical force.

### BRIEF DESCRIPTION OF DRAWING(S)

FIG. 1 illustrates two conformational states of oligo-NIPAm-20a.

FIG. 2 illustrates the radius of gyration vs. temperature of an exemplary oligo-NIPAm-20 embodiment.

FIG. 3 illustrates end-to-end distance d vs. compressing force F wherein Fc is the critical compression for an exemplary oligo-NIPMAm-30 embodiment.

FIG. 4 illustrates an Euler arch associated with certain aspects of presently disclosed embodiments.

FIG. 5 illustrates a bifurcation diagram of a cusp catastrophe model represented by distances d between the edges of the Euler arch vs pulling force F.

FIG. 6 illustrates an exemplary embodiment with a simulated applied force where one edge of a bent oligomeric chain is fixed, and a force F is applied to another edge of the chain.

FIG. 7 depicts time series of edge-to-edge distance of an exemplary NIPMAm-30 oligomer

FIG. 8 depicts spontaneous vibrations of an exemplary embodiment oligo-NIPMAm-30 (top panel) and statistical weights for visits of the “open” and “close” states when the pulling force F passes through a critical value.

FIG. 9 depicts stochastic resonance of an exemplary NIPMAm-30 oligomer controlled by a weak oscillating force.

FIG. 10 depicts a bistable system capable of spontaneously vibrating between two conformations.

FIG. 11 depicts time dependence of the edge-to-edge distances in an exemplary oligo-NIPMAm-30 embodiment in a vibratory regime near a critical pulling force.

FIG. 12 depicts statistics of visits to bent and stretched states normalized on the maximum value for an exemplary oligo-NIPMAm-30 embodiment near (left) and far from (right) a critical pulling force.

FIG. 13 depicts time dependence for the number of hydrogen bonds surrounding a hinge location of an exemplary NIPMAm-30 embodiment and edge-to-edge distance of the chain for pulling forces Fc=400 pN (left panel) and F=500 pN (right panel).

FIG. 14 depicts exemplary: (1400) Bistable oligomer with a single-molecule cargo attached to the oligomer surface; (1401) Oligo-NIPMAm-30 with an attached cargo molecule; a compressing force is applied to control spontaneous vibrations and stochastic resonance of the oligomer; and (1402-1405) Samples of tested molecular cargo: dye ATTO-390, hormones, amino acids tryptophan, estradinol, and triiodothyronine.

FIG. 15 depicts spontaneous vibrations of an exemplary oligomer with and without attached cargo molecules.

FIG. 16 depicts statistical weights distributions for visits of the “open” and “close” states of an exemplary oligomeric composition with a single tryptophan molecule vs a compressing force.

FIG. 17 depicts a shifting of the region of spontaneous vibrations of an exemplary oligo-NIPMAm-30 embodiment caused by attachment of different cargo molecules to the exemplary embodiment.

FIG. 18 depicts an exemplary embodiment wherein (1800) stochastic resonance of an oligo-NIPMAm-30 composition may be induced by a weak applied oscillatory force in a vibratory regime; and (1801) the attachment of a single tryptophan molecule shifts the vibratory regime and stochastic resonance may be transforms into ordinary forced oscillations.

FIG. 19 depicts an exemplary nanomachine embodiment that acts like a piston type engine comprising a bistable oligomeric machine connected to two nanotubes, one of which is reversibly moved inside the other.

FIG. 20 depicts unfolded (2000) and folded (2002) shapes of an exemplary NIPAm-20i embodiment.

FIG. 21 depicts the radius of gyration vs simulation time for an exemplary NIPMAm-30s embodiment.

FIG. 22 depicts unfolded (2200) and folded (2201) shapes of an exemplary NIPMAm-30s embodiment.

FIG. 23 depicts unfolded (2300) and folded (2301) shapes of an exemplary NIPMAm-30i embodiment.

FIG. 24 depicts radius of gyration vs simulation time for an exemplary NIPMAm 30i embodiment.

FIG. 25 depicts radius of gyration vs simulation time for an exemplary NIPAm-21i-19s di-block embodiment.

FIG. 26 depicts unfolded (2600) and folded (2601) shapes of an exemplary NIPAm-21i-19a di-block embodiment.

FIG. 27 depicts radius of gyration vs simulation time for and exemplary 21i-19a NIPAm di-block embodiment.

FIG. 28 depicts unfolded (2800) and folded (2801) shapes of an exemplary NIPAm-12i-4s-12i tri-block embodiment.

FIG. 29 depicts radius of gyration vs simulation time for an exemplary 12i-6s-12i NIPAm tri-block embodiment.

FIG. 30 depicts radius of gyration vs simulation time for an exemplary oligo-NIPAm-12i-8s-12i tri-block embodiment.

FIG. 31 depicts unfolded (3100), folded (3101), and semi-folded (3102) states of an exemplary NIPAm-12i-8s-12i tri-block embodiment.

FIG. 32 depicts unfolded (3200) and folded (3201) shapes of an exemplary oligo-NIPMAm-12i-7s-12i tri-block embodiment.

FIG. 33 depicts an exemplary 10-7-10-NMIPAm-NIPMAm-NMIPAm chimeric composition: (3300) and (3302) are the shapes of the chimeric composition in open and closed conformational states; (3301) temperature induced bistability of the chimeric composition; and (3103) spontaneous vibrations of the composition at T=320K.

FIG. 34 depicts an exemplary poly(para-phenylene) composition.

FIG. 35 depicts initial (open) (3500) and final (close) (3501) states of an exemplary nanoforceps embodiment.

FIG. 36 depicts distance between ends of rod-like fragments connected by PNIPAm vs time in an exemplary embodiment.

FIG. 37 depicts and exemplary embodiment of nanoforceps constructed with rod-like poly-(para-phenylene) fragments with 15 monomeric units and oligo-NIPAm-30s a power unit: left an oligo-NIPAm-30s in this exemplary embodiment is able to compress the rods with 33% degree of protonation; right an oligo-NIPAm-30s in this exemplary embodiment is not able to compress the rods with 50% degree of protonation.

### DETAILED DESCRIPTION

Oligomeric and/or molecular machines may include devices capable of exhibiting controlled movements at the nanoscale. Some oligomeric and/or molecular machines exhibit conformational bistability wherein these machines, under certain conditions, may be capable of changing between at least two conformations upon application of one or more stimuli. Some oligomeric and/or molecular machines may comprise various components such as oligomeric modules, bending and/or hinge regions, and extenders. Upon application or de-application of one or more stimuli, some molecular machines are configured to perform various mechanical motions such as vibrating, folding, bending, and/or extending. In some embodiments, mechanical motions may be utilized in various applications such as sensing, energy harvesting, drug delivery, and chemical reactions, among others.

### Bistable Oligomeric and/or Molecular Machines

Oligomeric machines may be configured to exhibit conformational bistability and may comprise oligomeric modules selected and joined so as to exhibit controllable and/or reproducible conformational changes in response to one or more stimuli. Conformational bistability may be characterized by the existence of at least two distinguishable conformational states wherein spontaneous or reproducible transitions between such states may be controllable by stimuli such as an externally controllable parameter. Non-limiting examples of conformational states include spatial shape or arrangement of a molecular, oligomeric, and/or polymeric material. For example, an oligomeric chain may have a stretched shape or it may be folded into a bent shape. Bistability implies that at least two conformational states are sufficiently stable or metastable for a desired process or application. For example, an oligomeric chain with a stretched state and a bent state may be repeatedly transitioned back-and-forth between the stretched state and bent state by stimuli. Oligomeric machines exhibiting conformational bistability may be utilized for nanomechanical operations. Nanomechanics refers to the movements performed by material structures such as, for example, molecular, oligomeric, and/or polymeric structures on the nanometer scale. The atomic fluctuations of such structures are typically much smaller than the structure size and its movements.

Currently, industrial miniaturization of devices and machines is carried out on the basis of top-down design. At the present, the scale of several tens of nanometers is industrially achievable. At the same time, it becomes clear that approaching the size of about 10 nm by top-down design, the cost of precise manipulations using “macroscopic” devices sharply increases and becomes too expensive in typical batch production. Alternatively, the manipulations by objects of a few nanometers in size should utilize “molecular machines” of approximately the same size. Nanomechanics enables machine-like movements at the nanometer scale using rigid nanoscale materials. Machine-like movement may imply the motion of a “solid” unit, i.e. the movements of rigid structures, wherein atomic fluctuations are much less than the structure's characteristic sizes and the scale of their movements. Since the atomic fluctuations at room temperature are of the order of 1 Angstrom, the minimal size of functional units will generally not be significantly less than 1 nanometer.

Oligomers comprise a few and/or many repeated monomeric units. Oligomers may comprise one or many types of monomeric units. For instance, oligomers may comprise one, two, three, or more types of monomeric units. The types of monomers may not be particularly limited so long as the oligomeric machines exhibit conformation bistability. For instance, monomers may comprise acrylamides, methacrylamides, acrylates, methacrylates, styrenics, alkenes, conjugated monomers, thiophenes, peptides, 2-Isopropyl-N-methylacrylamide, and/or peptoids. In preferred embodiments, monomeric unit comprise N-isopropylacrylamide (NIPAm) and/or N-isopropylmethacrylamide (NIPMAm). Some oligomers may comprise an isomer of NIPAm in which methyl and isopropyl groups are replaced one by another (NMIPAm). Oligomers may be synthesized by a variety of methods. The synthesis of oligomers is not particularly limited and some exemplary techniques include iterated synthesis, step growth syntheses, polymerization reactions, living polymerizations, living radical polymerizations, atom transfer radical polymerization, anionic polymerizations, cationic polymerizations, reversible addition fragmentation chain transfer polymerizations, ring open polymerization, metathesis reactions, and/or solid supported synthesis.

Oligomers may be synthesized in a single reaction or multiple reactions. Purification techniques may be used to fractionate and/or separate oligomers by, for example, molecular weight, functionality, tacticity, stereochemistry, and/or regiochemistry. Oligomers may contain more than one monomer type and may have various architectures such as block-co-oligomers, branched oligomers, random-co-oligomers, and/or gradient oligomers. Oligomers may be coupled together through a variety of means such as for instance click chemistry, azo-alkyne chemistry, thiol-ene chemistry, epoxy chemistry, Diels-Alder reactions, chain-end substitutions, and/or may be synthesized together in a single and/or multiple reaction steps. Oligomers may be telechelic. The tacticity may be controlled through various means such as, for example, though catalyst selection, solvent selection, reaction temperature, ligand selection, and/or polymerization reaction selection. The molecular weight of oligomers may be controlled by controlling reaction temperature, monomer concentration, initiator concentration, inhibitor concentration, reaction duration, post synthetic separation, and/or reactions may be quenched. Some exemplary embodiments include oligomers comprising N-isopropylacrylamide (NIPAm) and/or N-isopropylmethacrylamide (NIPMAm), Some exemplary embodiments include block-co-oligomers of N-isopropylacrylamide and/or N-isopropylmethacrylamide. Some exemplary embodiments include block-co-oligomers of N-isopropylacrylamide and/or N-isopropylmethacrylamide with one or more isotactic, atactic, and/or syndiotactic blocks. Some oligomers comprise at least 10, at least 15, at least 20, at least 25, and/or at least 30 monomeric units. Some oligomers have a persistence length of at least 0.5 nm, at least 1 nm, and/or at least 2 nm. Some oligomers may be at least 0.5 nm, at least 1 nm, at least 2 nm, at least 5 nm, and/or at least 10 nm long. Some oligomers may possess a lower critical solution temperature (LCST). Some oligomers may possess an upper critical solution temperature (UCST). Bulk poly(N-isopropylacrylamide) (PNIPAm) exhibits a LCST. The LCST of an oligomer may be different than the LCST of a longer polymer made from the same monomeric units. The LCST of an oligomer may be changed by changing the composition of the oligomer. The LCST of an oligomer may be changed by tuning the ratio of comonomers in an oligomer. Some oligomers may be polydisperse. Some oligomers may be monodisperse. Some oligomers may not possess any significant polydispersity. Some exemplary embodiments may comprise oligomeric fragments of PNIPAm of 20-30 units and PNIPMAm (poly-N-isopropylmethacrylamide) of the same length. Some embodiments comprise block-co-oligomer compositions with a central PNIPAm fragment of 5-15 units and two terminal PNMIPAm fragment of 5-20 units. Such exemplary embodiments may be configured to exhibit two clearly discernible conformational states, one of which corresponds to an unfolded, stretched form of oligomeric fragment, while the other has a folded, bent form. Transitions between these conformational states in these exemplary embodiments implement mechanic-like nano-scale motions of the fragment parts.

Oligomeric modules may be joined together. Oligomeric modules may be joined together during the synthesis of the oligomeric modules. Oligomeric modules may be joined together in a subsequent reaction. Oligomeric modules may be joined together at a bending and/or hinge region. The bending and/or hinge region may be inherent to the oligomeric structure. The bending and/or hinge region may comprise an additional molecular and/or oligomeric structure. The bending and/or hinge region may comprise a residue product from a linking reaction such as, for example, a click reaction, chain-end modification reaction, a thiol-ene reaction, an azo-alkyne reaction, a Diels-Alder reaction, an epoxy reaction, a esterification reaction, and/or a cyclo-addition reaction. A bending and/or hinge region may be flexible. A bending and/or hinge region may comprise, for example, acrylamide residues, methacrylamide residues, ether linkages, ethylene oxide units, peptides, and/or peptoids.

Oligomeric machines may be configured to exhibit conformational bistability and may comprise oligomeric modules selected and joined so as to exhibit controllable and/or reproducible conformational changes in response to one or more stimuli. Such stimuli may comprise, for example, one or more of a change in temperature, a set temperature, an electric field, a magnetic field, a change in pH, an applied force of at least 10 picoNewtons, a prescribed amount of energy, addition of a compound capable of associating with and/or binding the oligomeric machine, a change of solvent and/or co-solvent, and/or a change in ionic strength. One or more stimuli may induce fluctuations back-and-forth between a first and second conformation. One or more stimuli may induce a transition from a first conformation to a second conformation. One or more stimuli may induce a transition from a first conformation to a second conformation, and one or more additional stimulus may induce a transition from the second conformation back to the first conformation. One or more stimuli may induce a transition from a first conformation to a second conformation, and upon cessation of the one or more stimuli, the conformation may transition back from the second conformation to the first conformation. Oligomeric machines may be configured to vibrate stochastically at or near a transition point such as a transition temperature. Oligomeric machines may be configured to vibrate stochastically at or near a critical load and/or power load. A transition temperature may be between 250 K to 400 K, 275 K to 375 K, and/or 300 K to 350 K. A change in pH may be an increase in pH or a decrease in pH. A change in temperature may be an increase in temperature or a decrease in temperature. Some exemplary embodiments may comprise oligomeric N-isopropylacrylamide (oligo-NIPAm) with a length of about 10-15 monomeric units, oligomers comprising an isomer of NIPAm in which methyl and isopropyl groups are replaced one by another (NMIPAm), oligomeric poly-N-isopropylmethacrylamide (oligo-NIPMAm), and/or block-co-oligomers. In some embodiments, nanomechanical motion of a structural element may be realized by initiating a transition between an open conformation and a folded or closed conformation accompanied by movement of rigid molecular fragments. This phenomenon is different from coil-to-globule phase transitions in some PNIPAm polymers. Some exemplary embodiments show the advantages of conformational bistability of rather short oligomers together with reproducibility and mechanic-like motion.

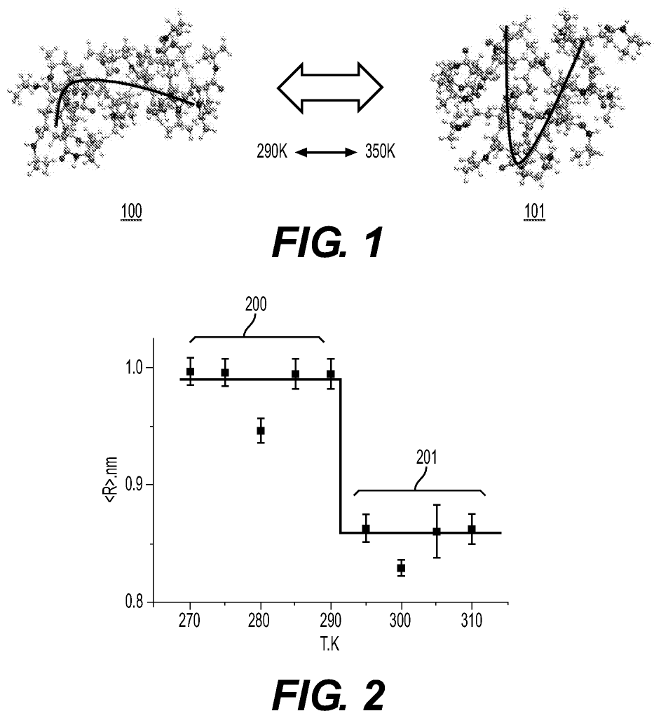

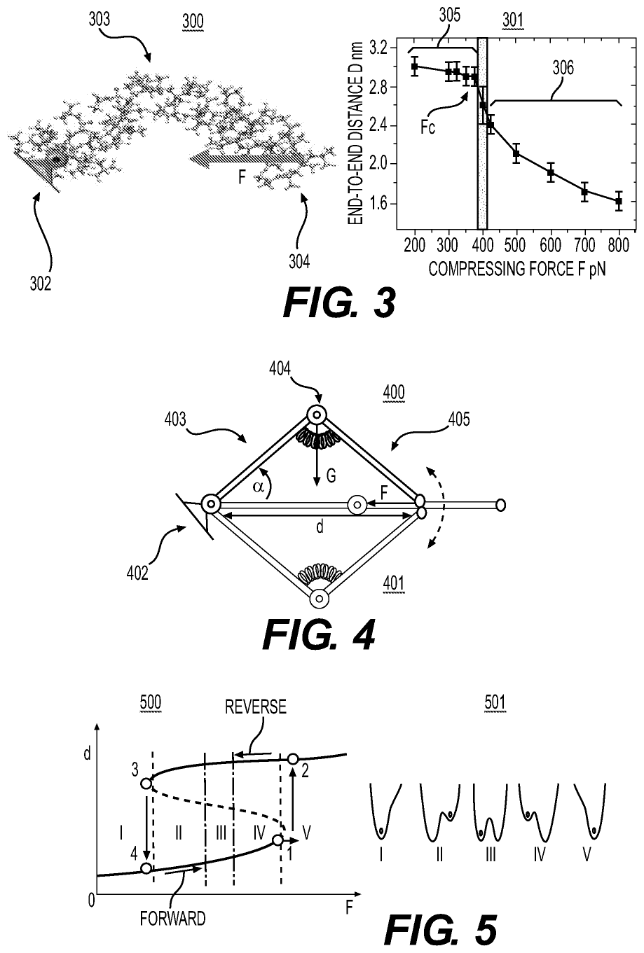

FIG. 1 illustrates an exemplary embodiment comprising two distinguishable conformational states of poly-N-isopropylacrylamide of 20 units in length (oligo-NIPAm-20) with temperature-controlled transitions between an open (100) and a closed (101) conformational state. Some embodiments may be configured as a nanomechanical power unit comprising two structural elements of oligo-NIPAm with a persistent Kuhn segment of about 1 nanometer. FIG. 2 illustrates a temperature-controlled transition between an open (200) and a closed (201) state of an exemplary oligo-NIPAm-20 at 290 K. FIG. 3 illustrates control of conformational transitions in an exemplary embodiment comprising oligo-NIPMAm-30 (element 303) wherein compressive forces (304) are applied to the ends of the oligo-NIPMAm-30 with one end fixed (302). In this exemplary embodiment, under compression close to 400 pN the open conformational state (305) becomes unstable and the oligomer sharply transits to the closed state (306).

In some embodiments, mechanical properties may be similar to the action of classical nonlinear mechanical systems such as an Euler arch or Zeeman's catastrophe machine. Catastrophe machines are mechanical devices with dynamics that demonstrate the “catastrophes”. FIG. 4 depicts an

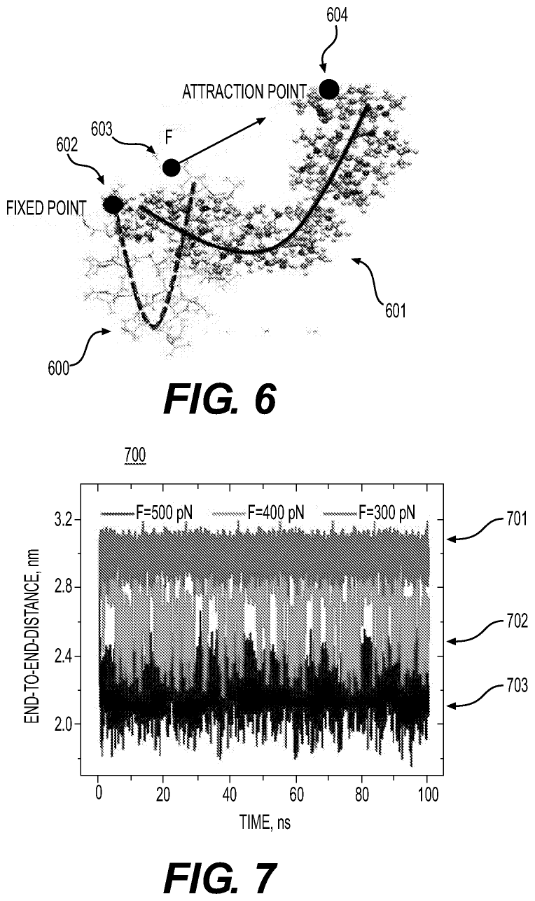

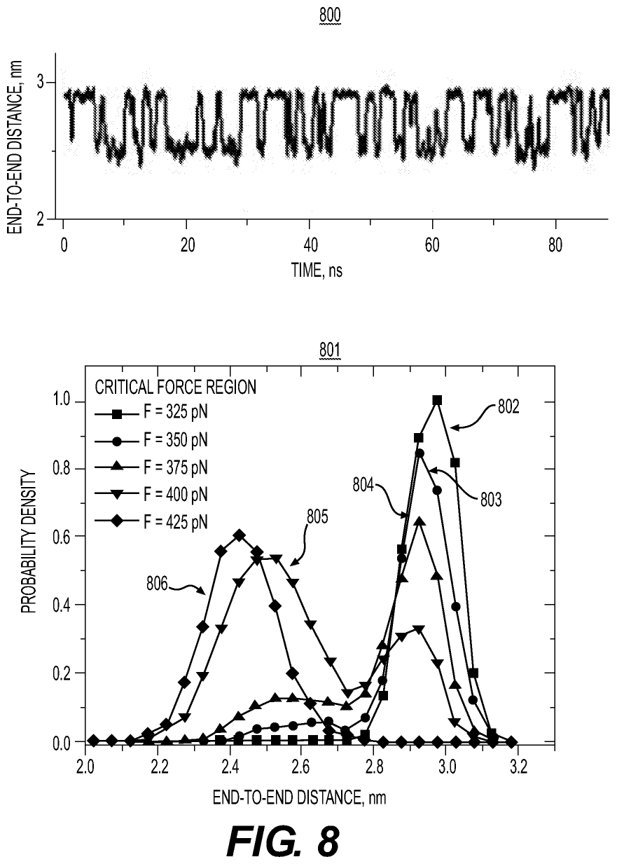

Euler arch which is one of the simplest mechanical constructions with “catastrophic” behavior and consists of two rigid rods (403 and 405) joint by an elastic hinge (404). To demonstrate the “catastrophes”, one edge of an Euler arch is fixed (402) and another edge is compressed by an external force. As the compressing force reaches a critical value, the Euler arch abruptly straightens. When the compressing force increases, the Euler arch shows bistablity with jump-like transitions from a stretched state into a bent state (400 and 401 depict two different bent states). For small pulling forces, the Euler arch remains stretched, however, as soon as the compressing force passes the critical value, the Euler arch is abruptly bent. In the theory of dynamical systems, such sharp changes are known as “catastrophes”. Accordingly, the Euler arch is referred as a “catastrophe machine”. The same catastrophes can be demonstrated by applying a pulling force to the bent arch. The bistablity of an Euler arch is described by a bifurcation diagram of the cusp catastrophe model as depicted by FIG. 5. In regions I and V of FIG. 5, the potential energy has a single minimum related to the bent and straightened Euler arch, respectively. In the regions II and IV, there are two energy minima, among which one of them dominates, while in the region III two local energy minima are symmetric and neither of the two states dominates. Some exemplary embodiments comprise nanoscale oligomeric machine components configured to exhibit “catastrophic” mechanical behavior. A preferred embodiment nanomechanical device that acts as a catastrophe machine may be an oligomeric composition consisting of two persistent Kuhn segments joint by a bending or hinge location. Such embodiments surprisingly demonstrate dynamical behavior of oligomeric compositions of a few nanometers in size. This may be demonstrated by two exemplary oligomeric compositions (oligo-NIPAm-20 oligo-NIPMAm-30) subjected to the action of a pulling force studied by computer simulation methods. GROMACS molecular dynamics package were used to perform atomistic simulations of the dynamics of oligomeric compositions in water at temperatures below and above the critical temperature of transition from the bent state to the stretched state. OPLS-AA force field in combination with TIP3P explicit water model are used to describe inter- and intra-molecular interactions. In such exemplary embodiments, an oligomeric composition in the bent (folded) conformation (600) at constant temperature is subjected to an applied force at one edge of the chain (603) and another edge of the chain was fixed (602) induce a transition into the straightened (unfolded) conformation (601). This configuration is depicted in FIG. 6. Varying the pulling force, F, that initiates the transition from the bent state to the straightened state, the threshold force is found to be about 400 pN (pico-Newton) for a NIPMAm-30 oligomer, and 120 pN for an oligo-NIPAm-20 oligomer. In FIG. 7, time series of edge-to-edge distance of a NIPMAm-30 oligomer are shown. Curve (701) correspond to the pulling force less than the threshold value, and curve (703) correspond to the pulling force greater than the threshold value. The pulling forces less than the threshold value do not stimulate the transition from the bent conformations to the stretched conformations. The forces greater than the threshold value stimulate the transition for rather short time. The dynamics of the compositions exhibit conformational bistability when the external force passes the threshold value. FIG. 8 demonstrates conformational bistability for some exemplary embodiments. In FIG. 8, curve (802) corresponds to a force of 325 pN, curve (803) corresponds to a force of 350 pN, curve (804) corresponds to a force of 375 pN, curve (805) corresponds to a force of 400 pN, and curve (806) corresponds to a force of 425 pN. In these exemplary systems near a threshold force, oligo-NIPMAm-30 and oligo NIPAm-20 alternately visit the bent and stretched states. Conformational bistability is demonstrated for small deviations, up to 20 pN, around 390 pN for oligo-NIPMAm-30 and 120 pN for oligo-NIPAm-20. For larger deviations, the oligomeric compositions have a well defied state, bent or stretched, respectively. Thus, bistability may be demonstrated by the dynamics exemplary embodiments. In this sense, oligo NIPMAm-30 and oligo-NIPAm-20 may be configured to exhibit “catastrophic” nanomechanical dynamics.

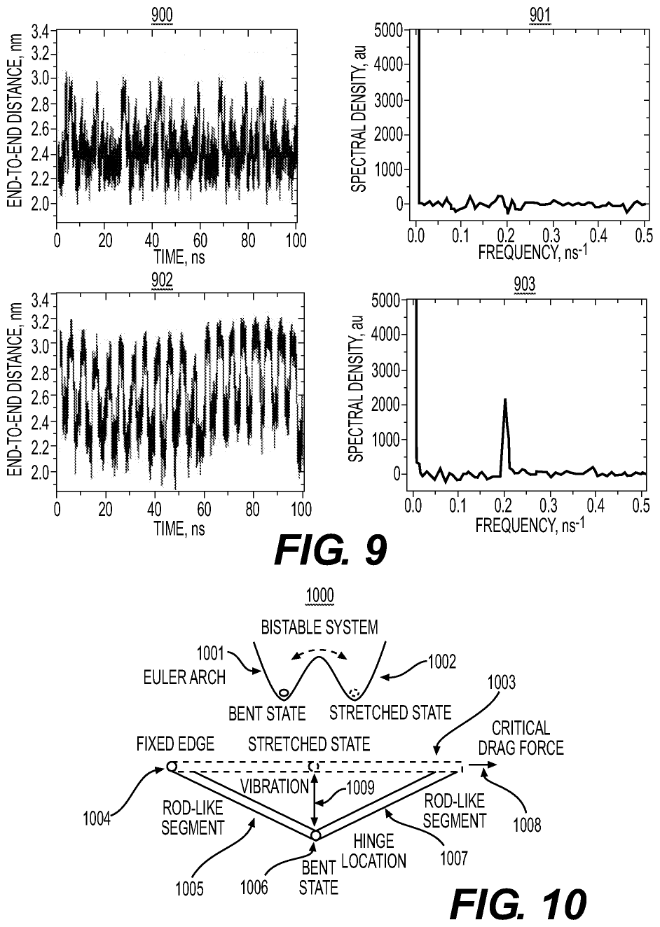

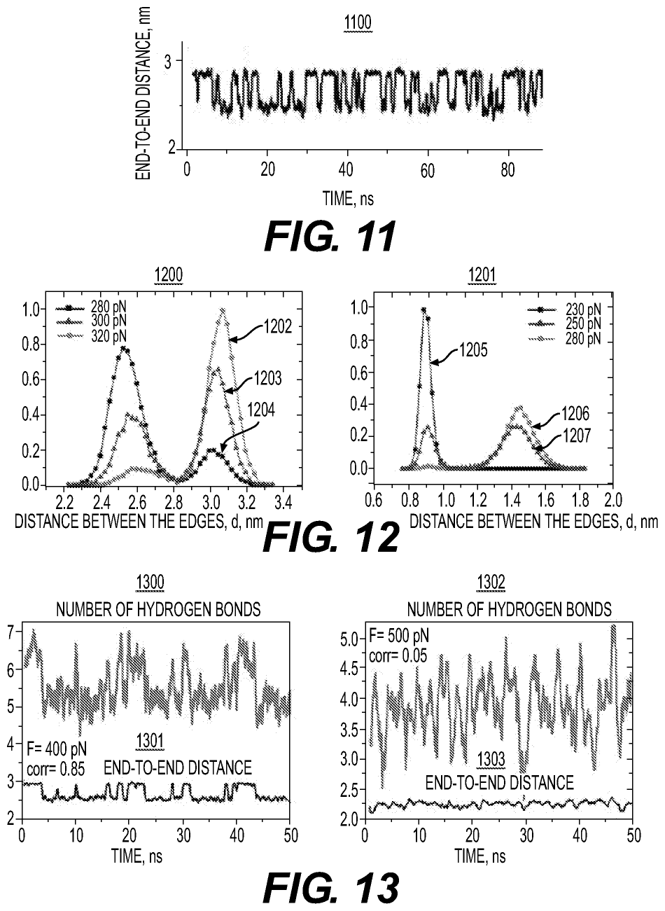

In some embodiments, cyclic variation of a control parameter near a threshold value may be demonstrated using full atomic computer simulations in a stochastic vibration regime by applying an additional weak oscillating force to simulate stochastic resonance. The cyclic variation of the pulling force near a threshold value Fc=400 pN by applying a weak oscillating electrical field with the amplitude E₀ ranged from 0.01-1.00 V/nm and the frequency varied from 50-500 MHz. Stochastic resonance was unambiguously observed under variation of these controlling parameters. An exemplary embodiment is illustrated in FIG. 9 wherein stochastic resonance of a NIPMAm-30 oligomer controlled by a weak oscillating force is depicted. Plots (900) and (901) show vibrations between two states and the frequency spectra of the vibrations in the case when the oscillation force is not applied to the oligomer. Plots (902) and (903) show a spontaneous resonance effect and the frequency spectra of the transitions in the case when a weak oscillation force controls the oligomer vibrations. In some embodiments, the nanometer scale makes it possible to directly access the bistability regime with thermal fluctuations. In some embodiments, a new class of nanomechanical devices, nanovibrators, may be constructed by using an unexpected effect of thermally activated vibration of bistable oligomeric compositions. FIG. 10 is a diagrammatic representation of this principle wherein (1000) depicts an energic profile of a bistable system which vibrates between a bent (1001) and stretched (1002) state. Elements (1003), (1004), (1005), (1006), (1007), (1008) and (1009) of FIG. 10 depict a stretched conformation, a fixed edge, a rigid element, a bending or hinge region, a rigid element, an applied force, and vibratory action respectively. A preferred nanomechanical embodiment may be an oligo-NIPMA-30 or oligo-NlPAm-20 compositions consisting of two persistent

Kuhn segments of about 1 nanometer in length joint by a bending or hinge location. The dynamics of oligomeric compositions subject to the action of pulling forces were studied by computer simulation methods. GROMACS molecular dynamics package were used to perform atomistic simulations of oligomeric compositions in water above a transition temperature. OPLS-AA force field in combination with TIP3P explicit water model are used to describe inter- and intra-molecular interactions. The dynamics of oligomeric compositions are characterized by the time dependence of the edge-to-edge distances in the chain. Thermally induced vibrations of the oligomeric compositions are established by fine-tuning control of the pulling force near a threshold value. FIG. 11 illustrates an exemplary embodiment and shows the time dependence of the edge-to-edge distances in oligo-NIPMAm-30 oligomer in a vibration regime near the critical pulling force Fc=400 pN. In the vicinity of a threshold value of pulling force for exemplary embodiments oligo-NIPMAm-30 and NIPAm-20, the oligomers alternately visit both the open and closed states. The vibration between these states occurs for rather small deviations of the pulling force, up to 10 pN, from 400 pN for oligo-NIPMAm-30 and from 120 pN for NIPAm-20 oligomer. FIG. 12 demonstrates for these exemplary embodiments that for larger deviations such as when the bistable potential is highly asymmetric, the oligomeric compositions are stuck in one of the two states and no vibration is produced. FIG. 12 depicts statistics of visits to bent and stretched states normalized on the maximum value for an exemplary oligo-NIPMAm-30 embodiment near (1200) and far from (1201) a critical pulling force respectively. Curves (1202), (1203), (1204), (1205), (1206), and (1207) correspond to forces of 280 pN, 300 pN, 320 pN, 230 pN. 250 pN, and 280 pN respectively. In some exemplary embodiments, non-covalent interactions may be used to modulate this bistable vibratory behavior. FIG. 13 illustrates an exemplary embodiment, where hydrogen bonding along the chain of oligo-NIPMAm-30 and between the oligo-NIPMAm-30 and surrounding water modulates the vibration. Surprisingly in these exemplary embodiments, no correlations between the number of hydrogen bonds surrounding the edge parts of NIPMAm-30 chain and the vibration is observed. FIG. 13 depicts an exemplary embodiment for NIPMAm-30 oligomer and shows time dependence of number of hydrogen bonds surrounding a hinge location in the top curves (1300 and 1302) and edge-to-edge distance of the chain in the bottom curve (1301 and 1303) for pulling forces of Fc=400 pN in the left panel (1300 and 1301) and F=500 pN in the right panel (1302 and 1303). Such embodiments show that the hydrogen bonds surrounding a hinge location of a NIPMAm-30 oligomer play a dominate role in the mechanic-like vibration of the oligomeric composition. In this embodiment, an oligomeric composition alternately visits two states with the time interval of about 5 nanoseconds in average, which corresponds to jumping over an activation barrier of about 10 k_{B}T and the vibrations are modulated by commutations of about 1 hydrogen bond in the hinge location area. Thermally induced vibrations reveal an important feature of some embodiments of molecular and/or oligomeric machines. The mechanic-like movement of such embodiments is well-distinguished from thermal fluctuations, but at the same time, the machine action may be activated even by low-potential thermal energy.

Oligomeric machines may comprise one or more extender elements. One or more extender elements may be attached to one end of a first oligomeric module. One or more extender elements may be attached to one end of a second oligomeric module. One or more extender elements may be attached to one end of a first oligomeric module and one end of a second oligomeric module. One or more extender elements may be attached to an oligomeric chain comprising one or more oligomeric modules. Extender elements may be rigid. An extender element may be a rigid molecular structure. Extender elements may be more rigid than the oligomeric modules. Extender elements may have a persistence length at least 1 nm, at least 2 nm, at least 5 nm, at least 10 nm, and/or at least 20 nm. An extender element may be, for example, a DNA fragment, a nanotube, an ionomer, a cationic polymer and/or oligomer, and/or an anionic polymer and/or oligomer. Extender elements may be attached to an oligomeric module via a covalent bond. Extender elements may be attached to an oligomeric module using, for example, click chemistry, a nitrene reaction, a thiol-ene reaction, an azo-alkyne reaction, a Diels-Alder reaction, a nucleophilic reaction, and/or an amide forming reaction. Extender elements may be polymerized off of an oligomeric initiator. Extender elements may comprise an initiator from which oligomeric elements are polymerized.

### Applications of Bistable Oligomeric and/or Molecular Machines

Oligomeric machines capable of exhibiting conformation bistability may comprise electric generating elements and may be configured to actuate an electric generating element. An electric generating element may be, for example, a piezoelectric element, a nanoparticle, a nanolayer, and/or a nanotube. An oligomeric machine may be configured such that as the oligomeric machine transitions from a first conformation to a second conformation, the oligomeric machine applies a stress to the electric generating element such as, for example, a piezoelectric element. The stress may include a compressive force, a tensile force, or a shear force. An oligomeric machine may be configured to apply a stress to an electric generating element in various ways. An oligomeric machine may be configured such that as the oligomeric machine transitions from a first conformation to a second conformation it applies a compressive stress to a piezoelectric element thus generating a voltage. An electric generating element may be attached covalently or noncovalently to an oligomeric machine. An electric generating element may be attached covalently or noncovalently to an oligomeric machine at a bending and/or hinge location. An electric generating element may be attached covalently or noncovalently to an oligomeric machine at a bending and/or hinge location such that as an oligomeric machine transitions from an open conformation to a closed or folded conformation, the oligomeric machine applies a stress to the electric generating element.

Oligomeric machines capable of exhibiting conformation bistability may comprise photo-absorbing elements and may be configured to change conformation upon absorption of light energy. Photo-absorbing elements may comprise, for example, one or more dye molecules, a conjugated molecule, an aromatic molecule, a semiconducting oligomer and/or polymer, a quantum dot, a nanoparticle, a stilbene moiety, an azobenzene moiety, and/or a bond configured for cis-trans isomerization. Oligomeric machines capable of exhibiting conformation bistability may comprise photo-absorbing elements at one or more bending and/or hinge regions. Oligomeric machines capable of exhibiting conformation bistability may comprise a bond configured for cis-trans isomerization at one or more bending and/or hinge regions such that upon absorption of light, the bond configured for cis-trans isomerization isomerizes thus inducing a conformational change of the oligomeric machine. A bond configured for cis-trans isomerization bond may be incorporated into an oligomeric machine, for example, by a polymerization reaction off of a bifunctional initiator comprising a bond configured for cis-trans isomerization.

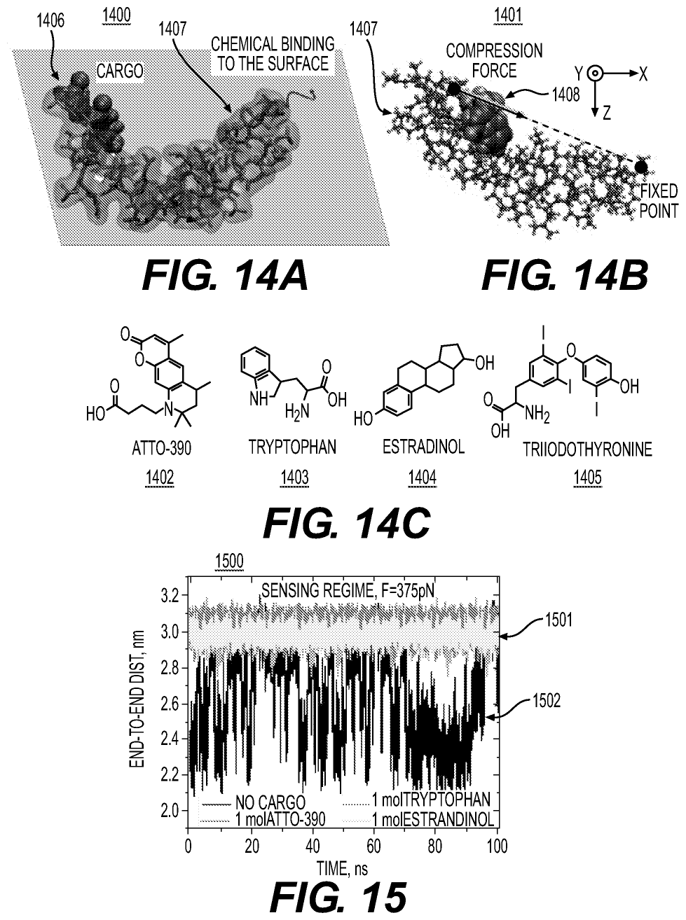

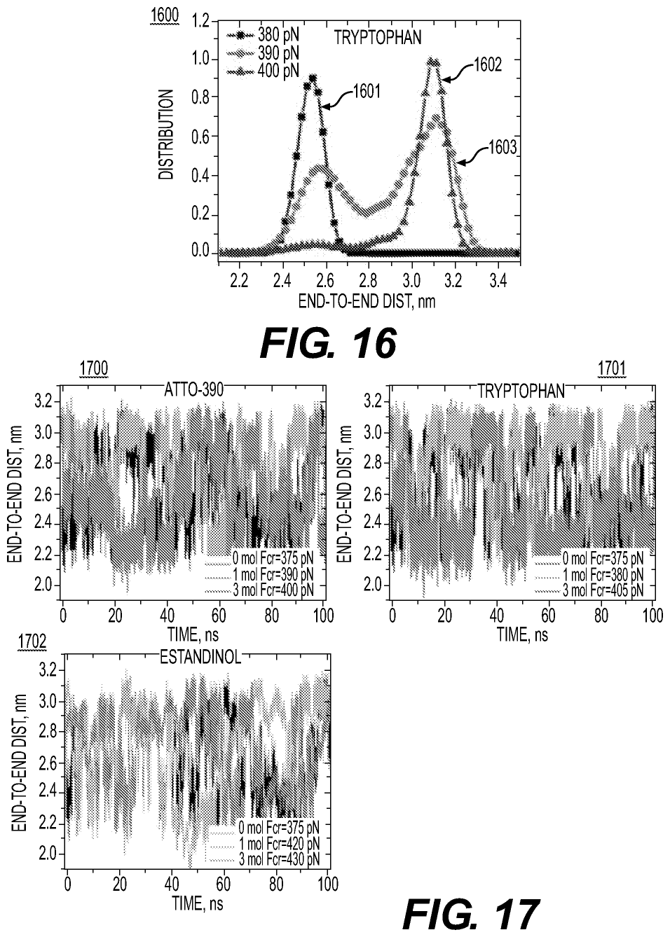

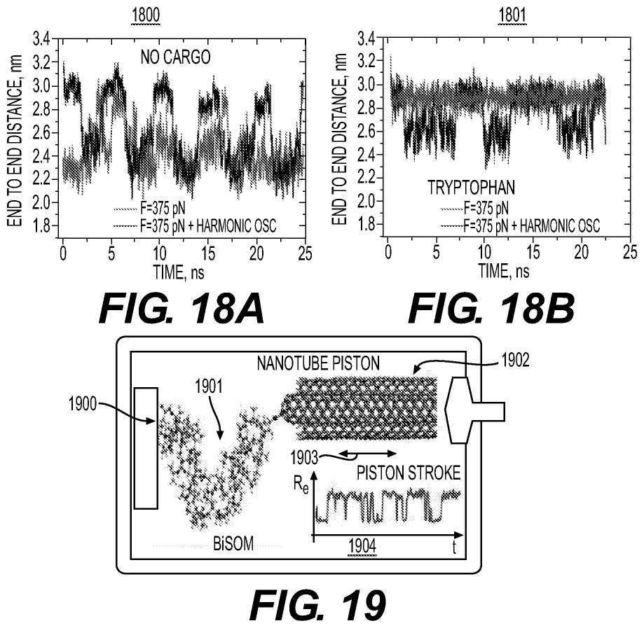

Oligomeric machines capable of exhibiting conformation bistability may be configured for sensing of analytes. Oligomeric machines may be configured such that upon binding and/or associating with an analyte, the oligomeric machine changes from a first conformation to a second conformation. Oligomeric machines may be configured such that upon binding and/or associating with an analyte, the frequency at which the oligomeric machine fluctuates between a first and second conformation is modulated. The frequency at which the oligomeric machine fluctuates between a first and second conformation may be decreased or increased. An analyte may be and/or comprise a small molecule, an amino acid, a saccharide, a hormone, an oligomer, a peptide, a metabolic product, a coordinating group, an ion, an aromatic group, a hydrogen bonding donor, and/or a hydrogen bonding acceptor. An oligomeric machine may comprise a Förster resonance energy transfer (FRET) donor and/or a FRET acceptor. An oligomeric machine may be configured such that a noncovalent interaction between a bending and/or hinge region of the oligomeric machine and an analyte induces a change in the conformation of the oligomeric machine cause a change in the spectroscopic properties of the oligomeric machine. Binding and/or association of an analyte with an oligomeric machine may modulate a FRET signal. Oligomeric compositions a few nanometers in size, which possess the property of conformational bistability associated with thermally activated spontaneous vibrations and stochastic resonance may be used as detecting units in the design of sensors. A physical mechanism of detection may be based on the sensitivity of spontaneous vibrations and stochastic resonance of the bistable oligomeric compositions to physical or chemical binding of an analyte to the oligomeric compositions. In an exemplary embodiment, spontaneous vibrations and stochastic resonance of two oligomeric compositions (oligo-NIPMAm-30 and oligo-NIPAm-20) subjected to attachment of molecular cargo were studied by computer simulation methods. An exemplary embodiment is illustrated in FIG. 14 wherein (1400) depicts a cargo molecule (1406 and 1408)) bound to an oligomeric machine component (1407). Exemplary cargo molecules include ATTO-390 (1402), Tryptophan (1403), estradinol (1404), and triiodothyronine (1405). GROMACS molecular dynamics package were used to perform atomistic simulations of the spontaneous vibrations and stochastic resonance dynamics of oligomeric compositions in water solution at temperatures below and above the critical temperature of transition from bent state to stretched state. OPLS-AA force field in combination with TIP3P explicit water model are used to describe inter- and intra-molecular interactions. The dynamics of oligomeric compositions were characterized by the time series of the distance between the chain edges. FIG. 15 and FIG. 16 depicts an exemplary embodiment wherein spontaneous vibrations of the oligo-NIPMAm-30 oligomeric composition respond to attachment of a cargo molecule. One can clearly see that, without cargo molecules, the spontaneous vibrations of oligo-NIPMAm-30 appear near the critical compression equal to about 375 pN. However, when a cargo molecule is attached to the oligomer subjected to the same compressive force, the oligomer completely leaves the vibrational mode. Single molecule detection by thermoactivated spontaneous vibrations of an exemplary oligo-NIPMAm-30 oligomeric composition is depicted in FIG. 15 and FIG. 16 wherein FIG. 15 depicts spontaneous vibrations of an oligomer with no cargo molecule attached (1502), it does not vibrate under these conditions when attaching a molecule (1501) of ATTO-390, tryptophan, and estradinol. In FIG. 16, statistical weights distributions for visits of the “open” and “close” states of the oligomeric composition with a single tryptophan molecule vs the compressing force is depicted for compressive forces of 380 pN (1601), 390 pN (1603), and 400 pN (1602) respectively. Bimodal distribution corresponding to the spontaneous vibrations mode. A control parameter such as, for example, compression force may be adjusted to a value at which spontaneous oscillations of the oligomer occur with cargo attached. FIG. 17 shows the shifting of the region of spontaneous vibration when cargo molecules are attached. When a cargo molecule attaches to an oligo-NIPMAm-30 oligomeric composition, the spontaneous vibration region shifts towards higher values of compressing force. For example, the spontaneous vibrations of the oligo-NIPMAm-30 oligomeric composition with the attached single tryptophan molecule occurs at about F_{c}^{tryp}=390 pN compressing force, while the spontaneous vibration of oligo-NIPMAm-30 itself occurs at about F_{c}^{tryp}=375 pN compressing force. The shifting of the region of spontaneous vibration when attaching cargo molecules suggests corresponding shifting of the stochastic resonance characteristics as depicted, for example, in FIG. 18. Shifts of the spontaneous vibrations and stochastic resonance modes may be sensitive to the number of detecting molecules, as well as to the molecule type. Detection units based on spontaneous vibrations of bistable oligomeric compositions are highly sensitive and may demonstrate a detection effect even for a single organic molecule.

Oligomeric machines may be configured for drug delivery. Oligomeric machines may comprise an imaging agent, a contrast agent, a therapeutic agent, and/or a theranostic agent. Therapeutic agents may comprise small molecule therapeutics, chemotherapeutics, and/or other therapeutic agents. Such therapeutic agents may be covalently bound to the oligomeric machine, noncovalently bound to the oligomeric machine, and/or encapsulated and bound to the oligomeric machine. Oligomeric machines may be configured for administration to a patient. A patient may be a human or non-human mammal. Oligomeric machines may be configured to modulate the biodistribution, pharmacodynamics, pharmacokinetics, and/or accumulation of the oligomeric machine in particular organs and/or tissues of a patient. Such configuration may comprise the enhanced permeability and retention (EPR) effect, antibody targeting, a peptide fragment, a peptoid fragment, and/or a nucleic acid fragment. An oligomeric machine may be configured for controlled release of a therapeutic agent. Controlled release of an agent may include solvolysis of a bond and/or a mechanical force which accompanies a conformational change. For example, a therapeutic agent may be encapsulated in polylactic acid and/or polyglactic acid. A therapeutic agent may be bound to an oligomeric machine through a hydrolysable bond such as, for example, an ester bond. An oligomeric machine may comprise a photo-absorber, such as a dye, quantum dot, and/or nanoparticle. An oligomeric machine comprising an absorber may generate localized heating upon absorption of light. An oligomeric machine comprising an absorber capable of generating localized heating upon absorption of light may be configured such that the local temperature change induces a change in conformation and/or frequency of conformational change in an oligomeric machine. An oligomeric machine may be configured such that a conformational change enables the oligomeric machine to grasp and/or bind a target structure, such as a molecule.

An oligomeric machine may be configured to induce, catalyze, and/or inhibit a chemical reaction. An oligomeric machine may be configured such that a conformational change brings two or more substrates into a preferred relative positioning for chemical reaction. An oligomeric machine may be configured to activate a bond for rupture. An oligomeric machine may catalyze bond formation. An oligomeric machine may be configured such that a change in conformation exposes and/or blocks a catalytic active site. An oligomeric machine may be configured such that a first conformation exposes a catalytic active site and a second conformation blocks a catalytic active site. A catalytic active site may comprise a transition metal catalyst and/or an organocatalyst, An oligomeric machine may comprise a catalytic triad wherein a first conformation is configure such that the catalytic triad is in a preferred conformation for catalytic activity and in a second conformation the catalytic triad is in a less preferred conformation for catalytic activity. An oligomeric machine may be configured such that a conformation change causes a bond to be stressed and/or more exposed. A stressed and/or more exposed bond may be activated to undergo further chemical reaction. For example, a stressed and/or more exposed ester bond may be more susceptible to hydrolysis.

An oligomeric machine may comprise a piston type element. A piston type element may be a rigid molecular structure such as a nanotube and/or a molecular structure possessing a persistence length greater that 10 nm. An oligomeric machine may be attached to a surface on one end and a rigid molecular structure on a second end. A oligomeric machine may be configured such that a change in conformation results in a mechanical actuation of a rigid molecular structure. An oligomeric machine may be attached to a surface using, for example, thiol chemistry, silane chemistry, and/or nitrene chemistry. An oligomeric machine may be synthesized on a solid support. An oligomeric machine may be attached to a rigid molecular structure using, for example, thiol chemistry, click chemistry, and/or nitrene chemistry. An oligomeric machine may be synthesized on a rigid molecular structure. An oligomeric machine may be synthesized on and/or attached to a solid support and may comprise an end functionalization configured to bind a rigid molecular structure. A rigid molecular structure may be configured to bind with and/or react with an end functionalized oligomeric machine. In some embodiments, oligomeric compositions of a few nanometers in size, which possess the property of conformational bistability, may be used as power units in an oligomeric machine. A nanomechanical device of the piston engine type with a bistable oligomeric composition that acts as a power unit is depicted in FIG. 19 wherein (1900) depicts an exemplary attaching point to a surface, (1901) depicts an exemplary oligomeric machine component, (1902) depicts an exemplary piston type element, (1903) depicts an exemplary actuation cycle, and (1904) depicts an exemplary radius of gyration vs time. Such an embodiment may comprise a complex composition constructed from three constructive elements. Two elements may be two nanotubes coaxially inserted one into the other like a piston in a cylinder. The inner nanotube is movable and acts as a piston, while the outer nanotube is stationary and acts as a cylinder. The third functional element of the nanomechaical device may comprise a bistable oligomer, one end of which is fixed, while the other end is connected to the inner nanotube of the piston-nanotube structure.

### Non-Limiting Exemplary Embodiments of Oligomeric and/or Molecular Machines and Applications Thereof

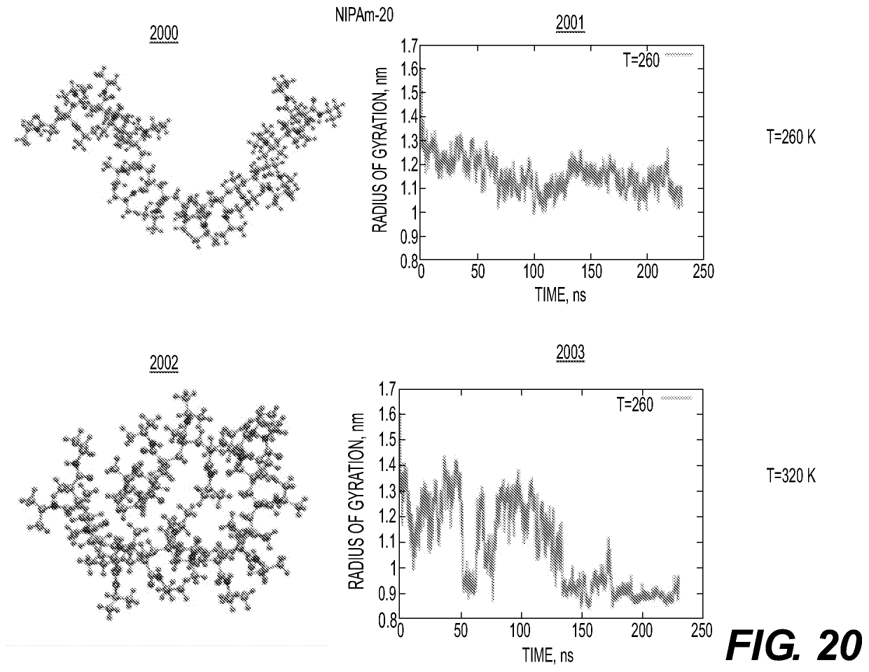

In some embodiments, PNIPAm oligomers, being of a length about two joint Kuhn segments, may undergo a reversible conformational change when the solute's temperature passes over LCST, thus reproducibly changing mutual orientation of the Kuhn segments in response to an external stimulus. This may be demonstrated using a series of computational experiments. Full atomic GROMACS molecular dynamics package were used to perform atomistic simulations of a NIPAm-oligomer in water solution at temperatures below and above LCST. OPLS-AA force field in combination with TIP3P explicit water model are used to describe inter- and intra-molecular interactions. The conformation of the chain is characterized by its radius of gyration and/or the distance between the chain ends. In an exemplary embodiment, an oligomer may comprise 20 NIPAm monomeric units connected isotactically (named oligo-NIPAm-20). FIG. 20 depicts an exemplary embodiment of oligo-NIPAm-20i wherein the chain is unfolded at the temperature of 280K with an average gyration radius of 1.15 nm (2000 and 2001), and it folds at the temperature of 320K to a state with an average gyration radius of 0.55 nm (2002 and 2003).

For some embodiments, 25-30 monomeric units connected syndiotactically is optimal, and it seemingly corresponds to approximately two persistent Kuhn segments. For instance, an oligomer consisting of 15 NIPAm monomers connected syndiotactically (named oligo-NIPAm-15s) does not show conformational bistability in response to temperature change, having gyration radius of 0.97 nm at 280K and 0.98 nm at 320K.

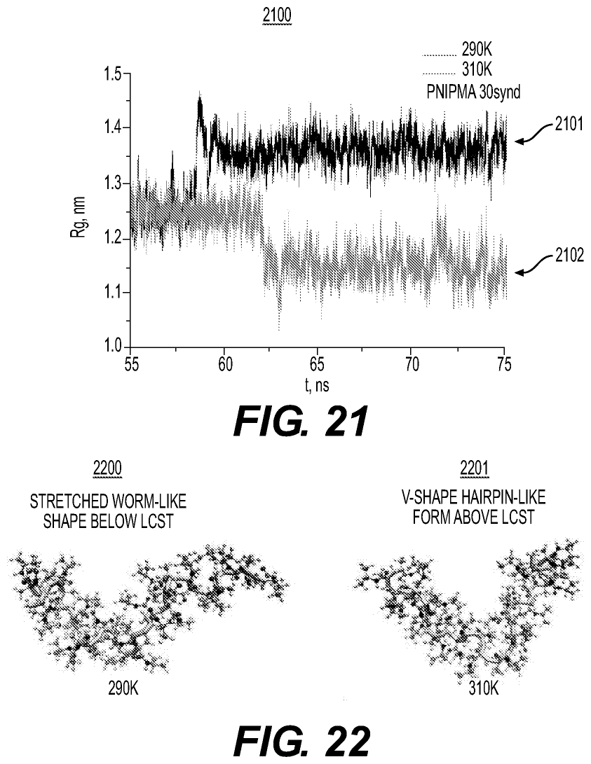

Poly-(N-isopropyl)-methacrylamide (PNIPMAm) is also a thermosensitive polymer that has an LCST at approximately 315K (42C/108F). In some embodiments PNIPMAm oligomers, being of a length about two joint persistent Kuhn segments, may be configured to undergo a conformational change from unfolded to folded states when a solute's temperature passes over the transition point. GROMACS molecular dynamics package was used to perform full atomistic simulations of single PNIPMAm oligomers in water at temperatures below and above LCST. OPLS-AA force field in combination with TIP3P explicit water model is used to describe inter- and intra-molecular interactions. The transition temperature for some oligomers is lower than the LCST for bulk polymers. In some embodiments, it is expected to be between 305K and 310K. FIG. 21 and FIG. 22 depicts an exemplary oligomer embodiment comprising 30 NIPMAm monomers connected syndiotactically (named oligo-NIPMAm-30s) and demonstrates an unfolded chain at the temperature of 290K with an average gyration radius of 1.35 nm (2101), and it folds at the temperature of 310K with an average gyration radius of 1.15 nm (2102). FIG. 22 depicts an exemplary oligomer's conformational change from worm-like unfolded shape (2200) to V-shaped hairpin-like folded structure (2200).

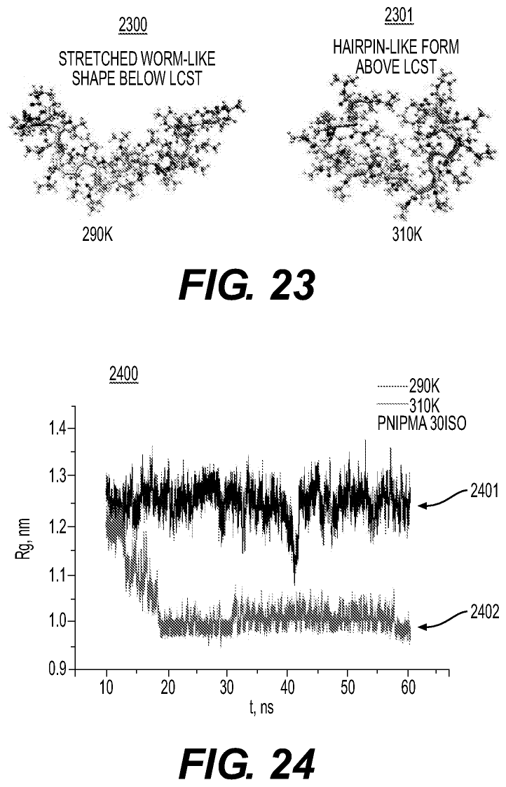

In an exemplary embodiment, FIG. 23 depicts an oligomer comprising 30 NIPMAm monomers connected isotactically (named oligo-NIPMAm-30i). In this embodiment, FIG. 24 shows that the chain is unfolded at the temperature of 290K with an average gyration radius of 1.37 nm (2401), and it folds at the temperature of 310K with an average gyration radius of 1 nm (2402). In this embodiment, the oligomer chain's conformation changes from stretched worm-like shape (2300) to folded hairpin-like shape (2301).

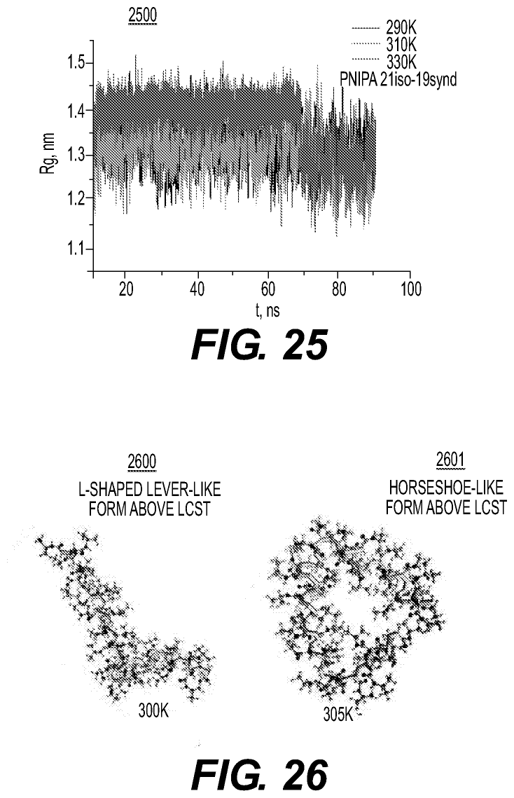

It may be demonstrated that some oligomeric structures do not demonstrate conformational bistability. For example, an oligomer consisting of a block of 21 NIPAm monomers connected isotactically, and a block of 19 NIPAm monomers connected syndiotactically (named oligo-NIPAm-21i-19s) does not demonstrate thermosensitive folding as shown in FIG. 25. In simulations performed on NIPMAm's oligomers consisting of 20 monomers connected atactically (named oligo-NIPMAm-20a) and syndiotactically (named oligo-NIPMAm-20s). Both oligomers do not show conformational bistability. At temperatures below and above LCST, they have average radii of gyration of 1 nm for oligo-NIPMAm-20a and 0.8 nm for oligo-NIPMAm-20s. An oligomer consisting of 30 NIPMAm monomers connected atactically named oligo-NIPMAm-30a, takes a stretched unfolded shape at the temperature of 290K with an average gyration radius of 1.57 nm, while at the temperature of 310K it has slightly more compact worm-like shape with an average gyration radius of 1.35 nm. Simulations were also performed on NIPMAm's oligomers consisting of 20 monomers connected atactically, named oligo-NIPMAm-20a, and syndiotactically, named oligo-NIPMAm-20s. Both oligomers do not show conformational bistability and at temperatures below and above the LCST, they have average radii of gyration of 1 nm for oligo-NIPMAm-20a and 0.8 nm for oligo-NIPMAm-20s.

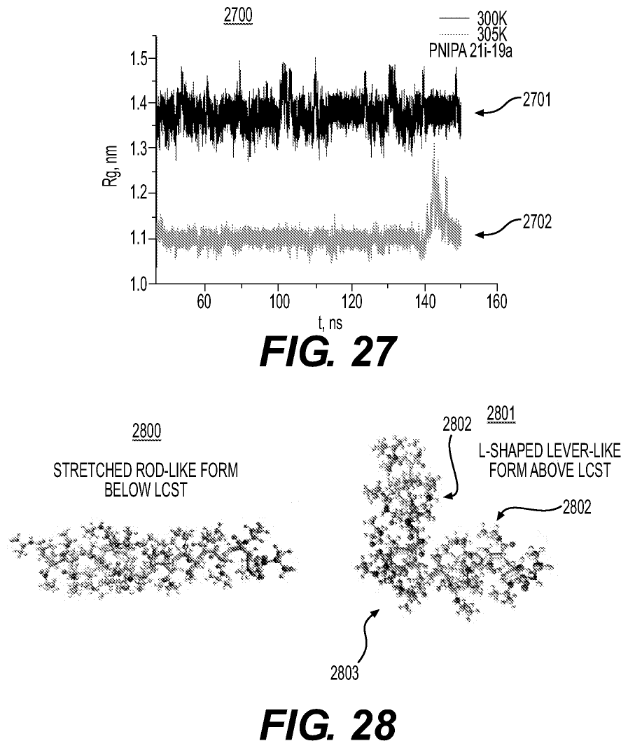

In some exemplary embodiments illustrated in FIG. 26, an oligomer may comprise two joined persistent blocks differing in tacticity and comprising a block of 21 NIPAm monomers connected isotactically and a block of 19 NIPAm monomers connected atactically (herein named oligo-NIPAm-21i-19a). This structure exhibits conformational bistability in response to temperature change wherein below a LCST it adopts an unfolded extended shape with an average radius of gyration of 1.37 nm (2600), and above LCST, the oligomer folds in to a horseshoe-like shape with an average radius of gyration of 1.1 nm (2601). FIG. 27 further illustrates this exemplary embodiment wherein small fluctuations in the radius of gyration relative to its variation in the folded (2702) and unfolded (2701) states show that these states are well defined.

In another exemplary embodiment, an oligomeric machine component comprising two stiff fragments of about 10 isotactic NIPAm units joined by a bending or hinge location of syndiotactic NIPAm is demonstrated. In a preferred three-block oligomeric composition comprising two edge blocks, each of 12 NIPAm monomers connected isotactically, which are joined by the bending location composed of 4 NIPAm monomers connected syndiotactically. This composition is denoted oligo-NIPAm-12i-4s-12i. Below the LCST, the oligo-NIPAm-12i-4s-12i composition exists predominately in a stretched rod-like structure with an average gyration radius of 1.3 nm. Above the LCST, this three-block oligomeric composition folds into an L-shaped lever-like form with an average radius of gyration of 1.05 nm. FIG. 28 depicts the oligo-NIPAm-12i-4s-12i composition which demonstrates two well-separated conformational states, unfolded (2800) and folded (2801) ones, with reproducible reversible transitions between the stretched form and the L-shaped lever-like form in response to external stimulus. In FIG. 28, element (2803) depicts a bending or hinge subcomponent, and elements (2802) depict rigid subcomponents.

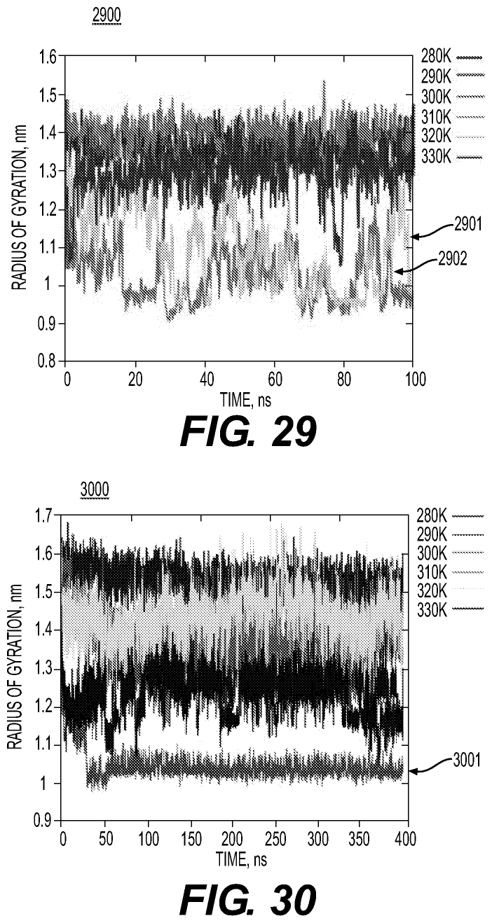

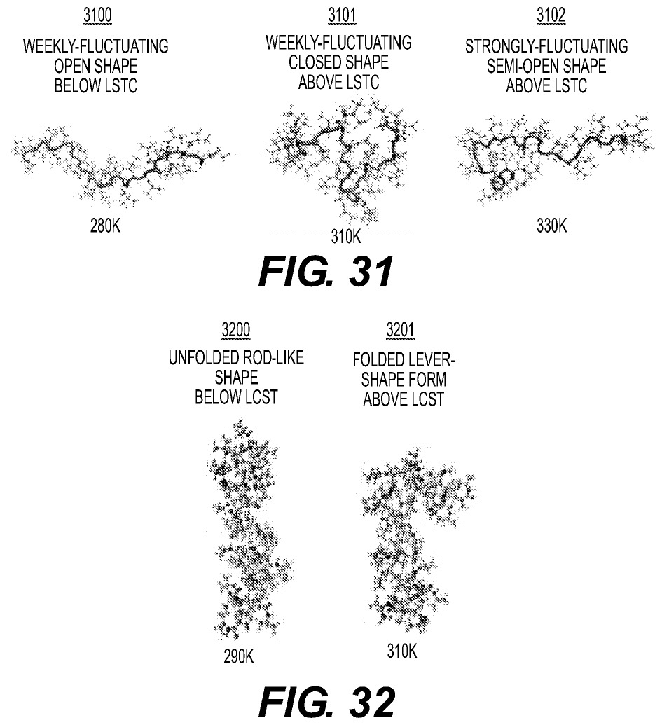

In another exemplary embodiment, a three-block oligomeric composition consists of two edge blocks, each of 15 NIPAm monomers connected isotactically are joined by a bending or hinge location composed of 10 NIPAm monomers connected syndiotactically and is named oligo-NIPAm-15i-10s-15i. Below LCST the composition exists as a stretched rod-like structure with an average gyration radius of 1.5 nm, while above the LCST it folds into a V-shaped hairpin-like form with an average radius of gyration of 1.25 nm. FIG. 29 depicts unfolded and folded shapes of oligo-NIPAm-15i-10s-15i three-block oligomeric composition. The oligo-NIPAm-15i-10s-15i composition also demonstrates two well-separated conformational states, unfolded and folded ones, with reproducible reversible transitions between the rod-like stretched form and the V-shaped hairpin-like form in response to external stimulus. Results of full atomic computer simulation of three oligo-NIPAm-compositions 12s-8i-12s, 9i-6s-9i and 12i-6s-12i are depicted in FIG. 30. Changing the order of blocks (that is, switching from 12i-8s-12i to 12s-8i-12s) leads to absence of bistability wherein the system has the same conformational state with an average radius of gyration of 1.25 nm at 290K and at 320K. Relatively short isotactic blocks (in 9i-6s-9i oligomer) seem to be less stiff due to lower probability of forming hydrogen bonds along the chain that sharply reduces the persistent length. As a result, the oligomeric composition folds to both 290K and 320K with an average gyration radius of 1 nm. Oligomeric composition named 12i-6s-12i shows different conformational states at temperatures below and above LCST, and the folded state strongly fluctuates. In some exemplary embodiments, other choices of three-block compositions may have two conformational states, but may exhibit less controllability in changes of mutual orientations of rigid edges of the chain when it folds in response to external stimulus. Such an exemplary embodiment may be a three-block oligomeric composition consisting of two edge blocks, each of 12 NIPAm monomers connected isotactically, which are joined by a bending location composed of 8 NIPAm monomers connected syndiotactically. This exemplary embodiment is named oligo-NIPAm-12i-8s-12i. FIG. 31 depicts a series of simulations at different temperatures with a step of 10K. At 280K the structure behaves as a stiff rod (3100). At 310K it collapses into well-folded S-shape state (3101). At 330K, due to high temperature, the entropy dominates over the hydrophobic interactions making the folded S-shape unstable (3102). Note that the composition's shape is unchanged over large simulation time. In some embodiments, additional three-block oligomers of NIPMAm are demonstrated. Isotactic fragments demonstrating high stiffness may be used as edge blocks, and syndiotactic fragments may bend and a syndiotactic fragment may be incorporated as a bending or hinge location. A preferred three-block oligomer comprising two edge blocks, each of 12 NIPMAm monomers connected isotactically may be joined by the bending location composed of 7 NIPMAm monomers connected syndiotactically and this exemplary embodiment is denoted by oligo-NIPMAm-12i-7s-12i. This oligomeric composition possesses conformational bistability with a transition temperature close to 300 K. Below the transition temperature it exists as a stretched rod-like structure with an average gyration radius of 1.26 nm, while above the transition temperature it folds into a Γ-shaped lever-like conformation with an average radius of gyration of 1.12 nm. As FIG. 32 depicts, an oligo-NIPMAm-12i-7s-12i composition demonstrates two well-separated conformational states, unfolded (3200) and folded (3201) ones, with reproducible reversible transitions between the stretched form and the Γ-shaped lever-like form in response to external stimulus.

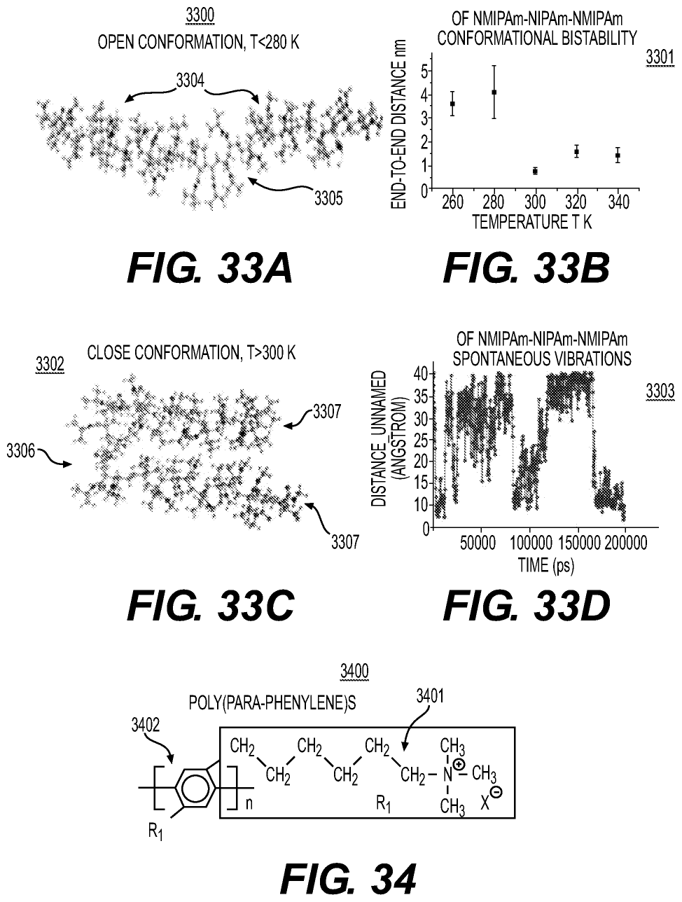

In another embodiment, three-block co-oligomers comprising two rigid blocks are joined by a bending third block. In some embodiments, oligo-NMIPAm fragments, a stereoisomer of NIPAm which methyl and isopropyl groups are replaced one by another, demonstrate high rigidness, and can be used as rigid edge blocks in the composition while oligo-NIPAm fragments may bend, and may be positioned at a bending or hinge location. For instance, a preferred three-block chimeric oligomer may comprise two edge NMIPMAm-blocks each of 10 monomers and are joined by the bending location composed of 7 NIPMAm monomers of isotactic configuration. The composition of this embodiment is denoted 10-7-10-NMIPMA-NIPMA-NMIPMA. In FIG. 33, elements (3305 and 3306) depict a bending or hinge subcomponent, and elements (3304 and 3307) depict rigid subcomponents. FIG. 33 (3300) depicts an open conformation for this embodiment. FIG. 33 (3302) depicts a close conformation for this embodiment. FIG. 33 (3301) depicts the conformational bistability exhibited by this embodiment. FIG. 33 (3303) depicts spontaneous vibrations exhibited by this embodiment at 320 K. This chimeric embodiment possesses a conformational transition above 300 K. Below the transition temperature it exists as an “open”, stretched structure with an average end-to-end distance about of 4 nm, while above the transition temperature it folds in half in to the “closed” conformation with the end-to-end distance about of 1 nm in average. Near the transition temperature, the 10-7-10-NMIPMA-NIPMA-NMIPMA composition demonstrates spontaneous transitions between the open and closed states, thus reproducibly changing mutual orientation of the rigid NMIPMAm fragments in response to external stimuli. A series of computational experiments were conducted on this embodiment. Full atomic GROMACS molecular dynamics package were used to perform atomistic simulations of NMIPMA-NIPMA-NMIPMA oligomers in water solution at temperatures ranged from 290K to 360K. OPLS-AA force field in combination with TIP3P explicit water model are used to describe inter- and intra-molecular interactions. The conformation of the chain is characterized by its radius of gyration and the distance between the oligomer ends.

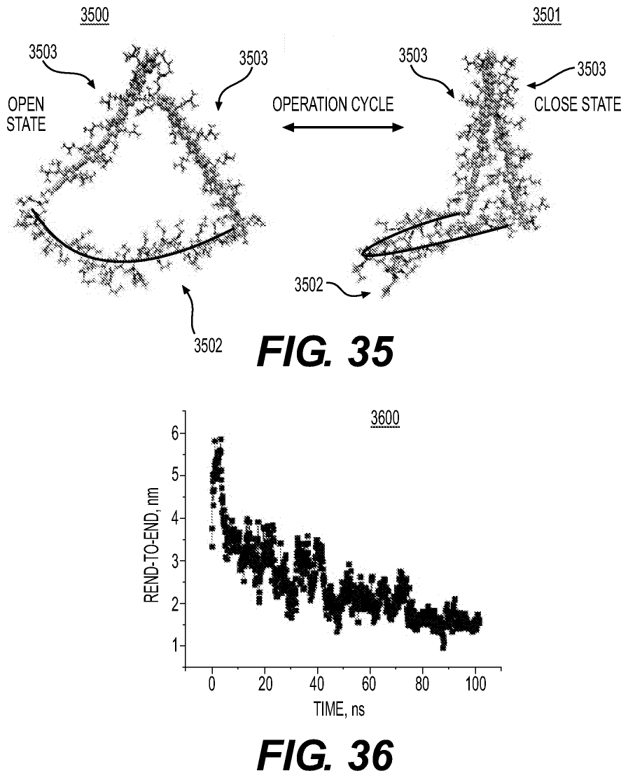

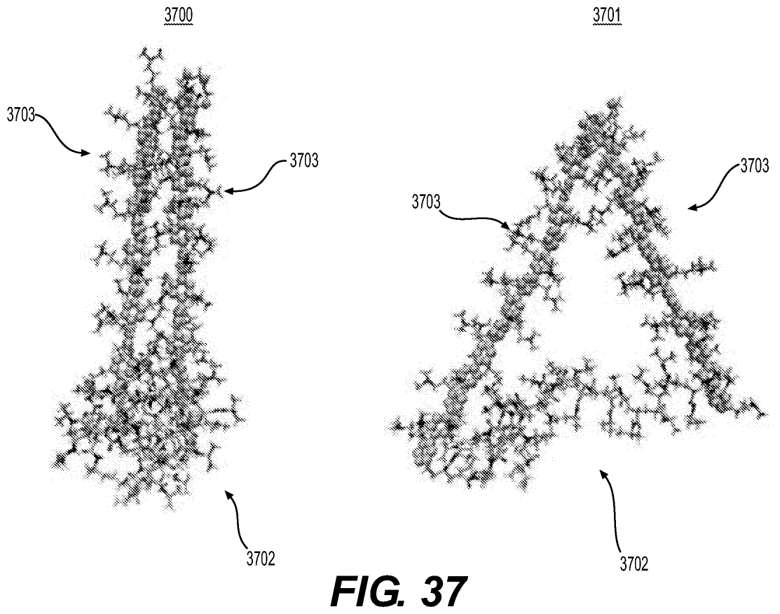

A nanomechanical device may be configured to act as a nanoforcep or molecular tweezer with an oligomeric composition that acts as a power unit. An exemplary embodiment nanomechanical device may comprise four oligomeric elements with two elements comprising poly(para-phenylene) rod-like segments of 10-20 monomeric units, and each phenyl ring in poly(para-phenylene) modified with short aliphatic chain with an amine group at its end Amine groups in the short aliphatic chains engrafted to the phenyl-rings may be protonated and/or deprotonated. FIG. 34 depicts and exemplary poly(para-phenylene) (3402) modified with aliphatic amine groups (3401). Changing a degree of protonation by changing pH of solvent, or using photoacids and/or photobases, may control electrostatic repulsion between the rod elements. A short (approximately 3 monomer) flexible polyoxyethylene fragment may tether the two poly(para-phenylene) elements close to each other, and a NIPAm-30s oligomer may be configured to act as a power unit. A distance between the edges rod fragments connected by a NIPAm-30s oligomer may vary due to the competition between electrostatic repulsion of rod segments and the mechanical compression force developed by NIPAm-30s during the conformational transition The NIPAm-30s oligomer, being able to undergo a reversible conformational transition, may provide sufficient mechanical force for compression of rod segments as depicted in FIG. 35. A series of computational experiments were conducted. Atomistic simulations of a NIPAm-30s oligomer in water solution at temperatures below and above the transition temperature of oligo-NIPAm-30s conformational bistability were performed using GROMACS molecular dynamics package. OPLS-AA force field in combination with TIP3P explicit water model were used to describe inter- and intra-molecular interactions. The action of this exemplary nanomechanical device is characterized by the distance between the edges of the NIPAm-30s oligomer. In some embodiments, poly(para-phenylene) rod segments of 10 monomeric units with 70% of protonated amine groups giving maximal distance between the rod-segments edges were connected by unfolded stretched PNIPAm-30s oligomer below the transition temperature of oligo-NIPAm-30s. FIG. 35 illustrates an exemplary embodiment wherein the temperature was set at 320K to induce a conformational transition of PNIPAm-30s. In FIG. 35, (3500) depicts exemplary nanoforceps in an open configuration, (3501) depicts a nanoforceps in an closed configuration, and elements (3503) depict rigid units comprising poly(para-phenylene), and (3502) depicts an oligomeric machine component. FIG. 36 demonstrates in this exemplary embodiment that NIPAm-30s oligomer may provide sufficient mechanical forces for compression of charged rod-like segments of modified poly-(para-phenylene) with 50% of protonated amine groups. The unfolded conformation of oligo-NIPAm-30s fragment transitions to the folded state. In this embodiment, the oligo-NIPAm-30s fragment compresses the rod-like fragments towards each other and against electrostatic repulsive interactions of the protonated amines. A variation of this embodiment with longer phenylene rod-like segments of 15 monomeric units may be demonstrated. Varying a degree of protonation in an exemplary embodiment demonstrates control of nanomechanical actuation in response to pH. FIG. 37 illustrates such an exemplary embodiment wherein a nanoforceps structure may be actuated at a lower degree of protonation of about 33%, but not at a higher degree of protonation of about 50%. In FIG. 37, (3700) depicts exemplary nanoforceps in an closed configuration, (3501) depicts a nanoforceps in an open configuration, and elements (3503) depict rigid units comprising poly(para-phenylene), and (3502) depicts an oligomeric machine component.

In a first non-limiting exemplary embodiment, two joined oligomeric modules possess conformational bistability with controlled conformational change. The molecular and/or oligomeric machine component comprises a first oligomeric module having a first end and a second end and a second oligomeric module having a first end and a second end. The first end of the first oligomeric module is joined to the first end of the second oligomeric module to form an oligomeric chain, and the second end of the first oligomeric module is disconnected from the second end of the second oligomeric modules. The first oligomeric module and the second oligomeric module are selected and joined so that a pair of joined oligomeric modules possesses conformational bistability. A relative orientation of the first oligomeric module and the second oligomeric module spontaneously changes from a first orientation to a second orientation in response to stochastic disturbance applied to the joined oligomeric modules. The relative orientation of the first oligomeric module and the second oligomeric module repeatedly changes from a first orientation to a second orientation in response to energy applied to the joined oligomeric modules. An oligomeric module may have a length between 0.5 nm and 20 nm. The relative orientation of the first and second oligomeric modules may define a conformation, and two such stable or metastable conformations may exist. A transition between a first conformation and a second conformation may include relative motion of the first and second oligomeric modules. An oligomeric module may comprise at least 5 repeat units. An oligomeric module may comprise at least 10 repeat units. An oligomeric module may comprise at least 15 repeat units. An oligomeric module may comprise at least 20 repeat units. An oligomeric module may comprise at least 25 repeat units. An oligomeric module may comprise at least 30 repeat units. An oligomeric module may comprise poly-N-isopropylacrylamide. An oligomeric module may comprise poly-N-isopropylmethacrylamide. An oligomeric and/or molecular machine component may be configured so as to exhibit a conformational transitional point within 250 K to 400 K. An oligomeric and/or molecular machine component may be configured so as to exhibit a conformational transitional point within 275 K to 375 K. An oligomeric and/or molecular machine component may be configured so as to exhibit a conformational transitional point within 300 K to 350 K. An oligomeric module may comprise a single monomeric unit. An oligomeric module may comprise NIPAm residues. An oligomeric module may comprise NIPMAm residues. The relative orientation of the first and second oligomeric modules may change in response to an application of light energy. The relative orientation of the first and second oligomeric modules may change in response to an application of an electric field. The relative orientation of the first and second oligomeric modules may change in response to an application of a magnetic field. The relative orientation of the first and second oligomeric modules may change in response to a reversible or irreversible chemical reaction. The relative orientation of the first and second oligomeric modules may change in response to a noncovalent interaction. The relative orientation of the first and second oligomeric modules may change in response to a ligand binding, ionic interaction, and/or hydrogen bonding. The relative orientation of the first and second oligomeric modules may change in response to a change in pH. The relative orientation of the first and second oligomeric modules may change in response to an application of heat and/or a change in temperature. The relative orientation of the first and second oligomeric modules may change in response to thermal fluctuations of the surroundings. The first and second oligomeric modules may be selected and joined so as to flex no more than 50% along a length of each oligomeric module. In some embodiments, flexure may be comparable with fluctuations of the atomic structure. In some embodiments, a difference between the first orientation and the second orientation of the oligomeric modules is the variation of distances between the disconnected ends of the modules of at least 0.2 nm. In some embodiments, a force of at least 10 picoNewtons may be applied to transition an oligomeric and/or molecular machine between a first and second conformation. In some embodiments, a transition between a first and second conformation may be configured to exert a force of at least 10 picoNewtons.

In a second non-limiting exemplary embodiment, sub-50 nm co-joined bistable oligomeric modules enable variable flexure. A bistable oligomeric machine may comprise a synthetic material including at least two co-joined oligomeric modules forming an oligomeric chain. Each oligomeric module may be a persistent oligomeric segment with a length from 0.5 nm to 15 nm. A bending and/or hinge element may be located at at least one position of co-joinder between the two co-joined oligomeric modules. The synthetic material may be selected such that an application of a prescribed amount of energy to the oligomeric chain causes the oligomeric modules to predictably flex relative to each other at the at least one bending or hinge location. The application of a prescribed amount of energy may include applying variable prescribed amounts of energy to cause a plurality of differing mechanical like motions. The plurality of differing mechanical like motions may include flexure in a first direction and reverse flexure in a second direction and/or opposite the first direction. A variable energy application may be binary and a value of one energy level is positive. A variable energy application may be binary and a value of one energy level is positive and another is approximately zero. The synthetic material may be selected such that application of a predetermined amount of energy to the oligomeric chain causes flexure about the bending or hinge location in a first direction, and wherein the application of one more portion of a predetermined amount of energy to the oligomeric chain causes a reversal of the flexure about the bending or the hinge location. The synthetic material may be selected such that application of a predetermined amount of energy to the oligomeric chain causes flexure about the bending or hinge location in a first direction and wherein cessation of application of the predetermined amount of energy to the oligomeric chain causes a reversal of the flexure about the hinge location. The synthetic material may be selected such that alternating application of energy levels causes repeated flexure at the bending or hinge location. The length of each oligomeric module may be between 0.5 nm and 20 nm. The bending or hinge location may be located at a weakened location in the oligomeric chain. The synthetic material may be an oligomer and/or polymer. The synthetic material may be selected such that application of a predetermined amount of energy to the oligomeric chain causes flexure about the bending or hinge location in a first direction and wherein cessation of application of the predetermined amount of energy to the oligomeric chain causes a reversal of the flexure about the hinge location. The synthetic material may be selected such that application of a predetermined amount of stochastic disturbance to the oligomeric chain causes spontaneous flexure about the bending or hinge location in a first direction and in the second direction and wherein cessation of application of the predetermined amount of stochastic disturbance to the oligomeric chain causes cessation of the spontaneous flexure about the hinge location. The synthetic material may be selected such that application of a predetermined amount of energy to the oligomeric chain causes flexure about the bending or hinge location in a first direction, and wherein the application of one more portion of a predetermined amount of energy to the oligomeric chain causes a reversal of the flexure about the bending or the hinge location. Oligomeric modules may predictably flex relative to each other at a at least one bending or hinge location. Some molecular and/or oligomeric machines may have variable and/or fixed lengths of Kuhn segments, and may not require that relative motion is repeatable.

In a third non-limiting exemplary embodiment, cyclical energy application and de-application causes cyclical oligomeric module flexure. A molecular and/or oligomeric machine may comprise a synthetic material including at least two co-joined oligomeric modules forming an oligomeric chain and at least one bending or hinge location at at least one position of co-joinder between the at least two co-joined oligomeric modules. The oligomeric chain may be formed such that in response to a prescribed amount of energy applied to the chain, a prescribed amount of relative flexure occurs between the at least two co-joined oligomeric modules about that at least one bending or hinge location to cause a change from a first orientation to a second orientation, and, upon cessation of the applied prescribed amount of energy, the at least two co-joined oligomeric modules return from the second orientation to the first orientation. The oligomeric chain may be formed such that in response to a repeated cyclical application and cessation of a prescribed amount of energy, the oligomeric chain flexes repeatedly from the first orientation to the second orientation. At least one of the co-joined oligomeric modules may comprise NIPAm residues. At least one of the co-joined oligomeric modules may comprise NIPMAm residues. At least one of the co-joined NIPAm oligomeric modules may be bound to a rigid molecular structure such as, for example, a nanotube or DNA. At least one of the co-joined NIPMAm oligomeric modules may be bound to a rigid molecular structure such as, for example, a nanotube or DNA. Cyclical application of energy may cause oligomeric modules to flex when energy is applied, and to return to an original position when energy is released or dissipated.

A fourth non-limiting exemplary embodiment may include bistable oligomeric machines with three connected portions, only the center one of which flexes. A molecular and/or oligomeric machine may comprise an oligomeric chain having a first portion, a second portion, and a third portion, wherein the second portion is located between the first potion and the third portion and the second portion has a flexibility substantially greater than flexibilities of each of the first portion and the third portion. The first portion and the third portion may be oligomeric modules and the second co-joined oligomeric portion may be configured such that when exposed to a prescribed amount of energy, the second portion is caused to flex while the first portion and the third portion remain substantially un-flexed. The first portion and the third portion may be oligomeric modules and the second co-joined oligomeric portion may be a pair of co-joined oligomeric modules configured such that when exposed to a prescribed amount of energy, the second portion is caused to flex while the first portion and the third portion remain substantially un-flexed. The molecules of the second oligomeric module may be chosen and arranged to predictably and repeatedly flex and return in response to application and removal of energy. A molecular and/or oligomeric machine may comprise three molecular segments arranged end to end with only the center segment being flexible.

In a fifth non-limiting exemplary embodiment, bistable oligomeric machines may include extender and flex portions formed of differing materials. A molecular and/or oligomeric machine component may comprise a synthetic material including at least a first oligomeric module and a second oligomeric module joined to the first oligomeric module to form a oligomeric chain, at least one bending or hinge location at a position of co-joinder between the first oligomeric module and the second oligomeric module, the bending or hinge location permitting relative flexure between the first oligomeric module and the second oligomeric module, and at least one extender attached to the joined oligomeric modules, the extender being formed of a material different from materials of the oligomeric modules. The material of the extender may be less flexible than the materials of the oligomeric modules. The extender may be connected to a distal end of the second oligomeric module. The extender may be formed of a material different from both the first oligomeric module and the second oligomeric module. The materials of the first oligomeric module and the second oligomeric module may be chosen such that upon application of a prescribed amount of energy to the oligomeric chain, relative flexure occurs at the bending or hinge location. The extender may have a length greater than a length of the second oligomeric module. The molecular and/or oligomeric machine may further comprise an additional extender connected to the first oligomeric module wherein the extender has a spiral configuration. An extender section may be attached on at least one end of a flexible oligomeric module to enhance functionality of a molecular and/or oligomeric machine. The extender may be formed of different materials than the flexible oligomeric module to allow for variability in length, rigidity, and/or chemical functionalization.

In a sixth non-limiting exemplary embodiment, bistable oligomeric machines may include Poly(N-isopropylacrylamide). A synthetic oligomer for a bistable oligomeric machine may comprise a fragment of Poly(N-isopropylacrylamide) (PNIPAm) of at least 15 repeating units. The PNIPAm-oligomer may be stereo-regular or stereo-irregular. The PNIPAm-oligomer may comprise regions which are isotactic, syndiotactic, and/or atactic. The PNIPAm oligomer may be such that at least a one-third portion of the PNIPAm oligomer does not flex more than 50% along a length of the at least one-third portion. The PNIPAm-oligomer may have conformational bistability with reproducible change of mutual displacement of the oligomer fragment ends in response to applied energy. The PNIPAm-oligomer may exhibit thermally activated spontaneous vibrations with reproducible change of mutual displacement of the oligomer fragment ends in response to applied stochastic perturbations. The PNIPAm-oligomer may exhibit stochastic resonance with reproducible change of mutual displacement of the oligomer fragment ends in response to applied energy. The PNIPAm-oligomer may be a block-co-oligomer. A block-co-oligomer may be composed of certain portions, each in isotactic, syndiotactic, or atactic form. A PNIPAm-co-oligomer may be selected such that each block of the PNIPAm-co-oligomer composition does not flex more than 50% along a length of the block. The PNIPAm-co-oligomer may have conformational bistability with reproducible change of mutual arrangement of the blocks in response to applied energy. The oligomer may comprise 20 units of NIPAm. The oligomer may comprise 25 units of NIPAm. The oligomer may comprise 30 units of NIPAm.

In a seventh non-limiting exemplary embodiment, bistable oligomeric machines may include Poly (N-isopropylmethacrylamide). A synthetic oligomer for a bistable oligomeric machine may comprise a fragment of Poly(N-isopropylmethacrylamide) (PNIPMAm) of at least 15 repeating units. The PNIPMAm-oligomer may be stereo-regular or stereo-irregular. The PNIPMAm-oligomer may comprise regions which are isotactic, syndiotactic, and/or atactic. The PNIPMAm oligomer may be such that at least a one-third portion of the PNIPMAm oligomer does not flex more than 50% along a length of the at least one-third portion. The PNIPMAm-oligomer may have conformational bistability with reproducible change of mutual displacement of the oligomer fragment ends in response to applied energy. The PNIPMAm-oligomer may exhibit thermally activated spontaneous vibrations with reproducible change of mutual displacement of the oligomer fragment ends in response to applied stochastic perturbations. The PNIPMAm-oligomer may exhibit stochastic resonance with reproducible change of mutual displacement of the oligomer fragment ends in response to applied energy. The PNIPMAm-oligomer may be a block-co-oligomer. A block-co-oligomer may be composed of certain portions, each in isotactic, syndiotactic, or atactic form. A PNIPMAm-co-oligomer may be selected such that each block of the PNIPMAm-co-oligomer composition does not flex more than 50% along a length of the block. The PNIPMAm-co-oligomer may have conformational bistability with reproducible change of mutual arrangement of the blocks in response to applied energy. The oligomer may comprise 20 units of NIPMAm. The oligomer may comprise 25 units of NIPMAm. The oligomer may comprise 30 units of NIPMAm.

An eighth non-limiting exemplary embodiment may include chimeric bistable oligomeric machines. A synthetic oligomer for a molecular and/or oligomeric machine may comprise a fragment of Poly(N-isopropylacrylamide) (PNIPAm) of at least 5 repeating units in the stereo-regular or the stereo-irregular form and at least one other oligomeric fraction other than PNIPAm of at least 0.5 nm in length and/or possessing a persistence length of at least 0.5 nm. At least one portion of the PNIPAm may be such that it does not flex more than 50% along a length of the at least one portion of the PNIPAm oligomer and does not flex more than 50% along a length of the at least one-third portion. The oligomer fragment may exhibit conformational bistability with reproducible change of spatial arrangements of the fragment ends in response to applied energy. The oligomer composition may include three oligomeric modules with two edge NMIPAm-modules each of 10 monomers which are joined by a bending location composed of 7 NIPMAm monomers of isotactic configuration. A chimeric composition may allow for significant customization of the structure and function of a molecular and/or oligomeric machine such as, for example, length, rigidity, and/or chemical functionalization. The oligomer composition may comprise three modules with two edge NMIPAm-modules each of 8 monomers, which are joint by the bending location composed of 5 NIPMAm monomers of isotactic configuration.

Another non-limiting exemplary embodiment may include bistable oligomeric machines configured for mechanical force generation. A molecular and/or oligomeric machine may comprise a synthetic material including a first oligomeric module and a second oligomeric module joined to the first oligomeric module to form an oligomeric chain, at least one bending or hinge location at a position of co-joinder between the first oligomeric module and the second oligomeric module, the bending or hinge location permitting relative flexure between the first oligomeric module and the second oligomeric module, at least one element of a piston type; and a substrate configured with the piston element and the oligomeric chain such that the second oligomeric module of the oligomeric chain is capable of mechanically actuating the piston element. The piston element may be any suitable rigid molecular and/or nanoscale structure such as for instance a graphene nanotube, a nano-wire, and/or a DNA fragment. A bistable oligomer machine may generate mechanical force by transmitting the movements of a bistable oligomer machine to cyclic movement of a piston type element.

Another non-limiting exemplary embodiment may include bistable oligomeric machines for electro-mechanical nano-devices. A molecular and/or oligomeric machine may comprise a synthetic material including a first oligomeric module and a second oligomeric module joined to the first oligomeric module to form an oligomeric chain, at least one bending or hinge location at a position of co-joinder between the first oligomeric module and the second oligomeric module, the bending or hinge location permitting relative flexure between the first oligomeric module and the second oligomeric module; at least one electric generating element, and a substrate configured with the electric generating element and the oligomeric chain such that the second oligomeric module of the oligomeric chain is capable of mechanically actuating the electric generating elements. The oligomeric chain may be formed such that in response to a prescribed amount of energy applied thereto, relative movement occurs between the first oligomeric module and the second oligomeric module in a manner causing the mechanical action of the second oligomeric module on the electric generating element to produce an electrical voltage and/or current. The electric generating element may be a piezoelectric element, a nano-particle, a nano-wire, and/or a nano-layer. A molecular and/or oligomeric machine may be configured to generate a voltage by performing a mechanical action on a piezoelectric element.

Other exemplary embodiments may include bistable oligomeric machines for energy harvesting. A molecular and/or oligomeric machine may comprise a synthetic material including a first oligomeric module and a second oligomeric module joined to the first oligomeric module to form an oligomeric chain, at least one bending or hinge location at a position of co-joinder between the first oligomeric module and the second oligomeric module, the bending or hinge location permitting relative flexure between the first oligomeric module and the second oligomeric module; at least one light-absorbing element attached to the oligomeric chain at the bending or hinge location, at least one electric generating element, and a substrate configured with the electric generating element and the oligomeric chain such that the second oligomeric modules of the oligomeric chain to ensure the mechanical action on the electric generating elements. The oligomeric chain may be formed such that in response to a prescribed amount of energy applied thereto, relative movement occurs between the first oligomeric module and the second oligomeric module in a manner causing the mechanical action of the second oligomeric module on the electric generating element to produce an electrical voltage and/or current. The electric generating element may be a piezoelectric element, a nano-particle, a nano-wire, and/or a nano-layer. A molecular and/or oligomeric machine may be configured to generate a voltage by performing a mechanical action on a piezoelectric element. A light absorbing element may be a dye, a compound comprising aromatic groups, a compound comprising conjugation, and/or a semi-conducting element.

Other exemplary embodiments may include bistable oligomeric machines for sensing. A molecular and/or oligomeric machine may comprise a synthetic material including a first oligomeric module and a second oligomeric module joined to the first oligomeric module to form an oligomeric chain, at least one bending or hinge location at a position of co-joinder between the first oligomeric module and the second oligomeric module, the bending or hinge location permitting relative flexure between the first oligomeric module and the second oligomeric module; at least one chemically specific site disposed in the bending or hinge location of the oligomeric chain for selective binding of a detectable molecule, at least one electric generating element, and a substrate configured with the electric generating element and the oligomeric chain such that the second oligomeric modules of the oligomeric chain to ensure the mechanical action on the electric generating elements. The oligomeric chain may be formed such that in response to a prescribed amount of energy applied thereto, relative movement occurs between the first oligomeric module and the second oligomeric module in a manner causing the mechanical action of the second oligomeric module on the electric generating element to produce an electrical voltage and/or current. The electric generating element may be a piezoelectric element, a nano-particle, a nano-wire, and/or a nano-layer. The oligomeric chain with a chemically specific site may be formed such that in response to binding of a detectible molecule, relative movement occurs between the first oligomeric module and the second oligomeric module in a manner causing the pressure of the second oligomeric module on the electric generating element to produce an electrical voltage. A synthetic material comprising a first oligomeric module and a second oligomeric module joined to the first oligomeric module to form an oligomeric chain may possess thermally activated spontaneous vibrations with reproducible change of mutual displacement of the oligomeric modules in response to stochastic perturbations. The detecting unit may comprise a NIPAm-20 oligomeric composition. The detecting unit may comprise a NIPMAm-30 oligomeric composition.

Additional exemplary embodiments may include bistable oligomeric machines configured for drug encapsulating and delivery. A molecular drug delivery machine may comprise a first oligomeric module, a second oligomeric module connected to the first oligomeric module at a bend or hinge location and forming an oligomeric chain, and a therapeutic agent captured between the first oligomeric module and the second oligomeric module. The oligomeric chain may be configured such that upon application of energy thereto, relative movement occurs between the first oligomeric module and the second oligomeric module in a manner releasing the captured therapeutic agent. The therapeutic agent may be captured between the first oligomeric module and the second oligomeric module in a manner impeding degradation of a therapeutic agent. The therapeutic agent may have a lower affinity for at least one organ in a mammalian body than the first and second oligomeric modules, thereby enabling accumulation of the therapeutic agent in the at least one organ. The molecular and/or oligomeric machine may further comprise a targeting agent which targets delivery of the therapeutic agent to at least one organ in a mammalian body. The therapeutic agent may be captured between the first oligomeric module and the oligomeric module segment by a covalent and/or non-covalent bond between the therapeutic agent and the molecular drug delivery machine. The non-covalent bond may be a mechanical bond, a van der Waals bond, and/or a hydrogen bond. The therapeutic agent may be captured between the first oligomeric module and the second oligomeric module by a chemical bond between the therapeutic agent and the oligomeric drug delivery machine. The chemical bond may be a hydrolysable bond and when relative movement occurs between the first oligomeric module and the second oligomeric module the hydrolysable bond is exposed to a solvent, thereby breaking the hydrolysable bond and releasing the captured therapeutic agent. The oligomeric chain may be configured such that upon application of heat thereto the therapeutic agent is released. The therapeutic agent may be a negative regulator of a mammalian heat shock response, such that the release of the therapeutic agent reduces a mammalian heat shock response after application of heat. The therapeutic agent may be an agent that induces mammalian cell death. A molecular and/or oligomeric machine may be configured for precise drug delivery and release at a target location. For example, a therapeutic agent may be encapsulated within a bistable oligomeric machine, which may release the therapeutic agent upon actuation of the oligomeric machine.

Still other non-limiting exemplary embodiments may include bistable oligomeric machines for controlled release of a therapeutic agent. A molecular drug delivery machine may comprise a first oligomeric module, a second oligomeric module connected to the first oligomeric module at a bend or hinge location and forming an oligomeric chain, an encapsulation structure arranged for engagement by the oligomeric chain, and a therapeutic agent within the encapsulation structure. The oligomeric chain may be configured such that upon application of energy thereto, relative movement occurs between the first oligomeric module and the second oligomeric module in a manner causing engagement with the encapsulation structure in a manner causing rupture of the encapsulation structure and release of the therapeutic agent. The therapeutic agent may comprise at least two molecules. The molecular and/or oligomeric machine may comprise an additional therapeutic agent for simultaneous delivery with the therapeutic agent within the encapsulation structure. The molecular and/or oligomeric machine may comprise at least one targeting agent for targeting delivery of the therapeutic agent to at least one organ in a mammalian body. The molecular and/or oligomeric drug delivery machine may be configured for irreversible destruction upon rupture of the encapsulation structure, thereby allowing components of the encapsulation structure to be metabolized by a mammalian body. The encapsulation structure may comprise at least two oligomeric chains. A molecular and/or oligomeric machine may be configured to break open a vesicle containing a therapeutic agent.

Other non-limiting exemplary embodiments may include thermally activated bistable oligomeric machines. A molecular and/or oligomeric machine may comprise an arrangement of molecules configured for introduction into a mammalian body, the molecules being arranged and selected such that when exposed to a prescribed temperature, the arrangement performs at least one mechanical function selected from a group consisting of vibrations, extending, rotating, lifting, pressing, ratcheting, springing, and flexing. The prescribed temperature may be a normal mammalian body temperature and is below a temperature causing necrosis to mammalian cells. The molecular and/or oligomeric machine may be configured such that upon introduction of the molecular arrangement to the mammalian body, the molecular arrangement does not perform the mechanical function until the arrangement of molecules is exposed to a temperature at least equal to the prescribed temperature. The molecular arrangement may comprise polymeric material. The molecular and/or oligomeric machine may comprise a sensitizer configured to increase a local temperature. The sensitizer may be chemically attached to the arrangement of molecules. The sensitizer may comprise at least one nanoparticle and/or organic dye. The sensitizer may be configured to absorb light in the infra-red spectrum. The molecular and/or oligomeric machine may be configured as a drug delivery machine. The molecular and/or oligomeric machine may be chemically attached to a payload, and the molecular and/or oligomeric machine may be configured as a molecular shuttle to move the payload. The molecular and/or oligomeric machine may be configured as a molecular linear actuator. The molecular and/or oligomeric machine may be configured as a molecular sensor. The molecular and/or oligomeric machine may comprise at least one imaging agent that activates after the molecular and/or oligomeric machine performs a mechanical function. The molecular and/or oligomeric machine may be configured as a molecular tweezer wherein at least one mechanical function contracts at least two distal ends of the molecular and/or oligomeric machine to grasp an object between the distal ends of the molecular and/or oligomeric machine.

Still other non-limiting exemplary embodiments may include oligomeric machines configured to facilitate a chemical reaction. A molecular and/or oligomeric machine may comprise a synthetic material including a first oligomeric module and a second oligomeric module joined to the first module to form an oligomeric chain, at least one bend or hinge location at a position of co-joinder between the first oligomeric module and the second oligomeric module, the bend or hinge location permitting relative flexure between the first oligomeric module and the second oligomeric module, a first chemical reagent attached to the first oligomeric module, and a second chemical reagent attached to the second oligomeric module. The oligomeric chain may be formed such that in response to a prescribed amount of energy applied thereto, the first chemical reagent and the second chemical reagent are caused to be drawn into contact with each other and to undergo a chemical reaction. The oligomeric chain may be formed such that in response to a prescribed amount of energy applied thereto, the first chemical reagent and the second chemical reagent are caused to be drawn into contact with each other and to undergo a chemical reaction. The chemical reaction may comprise a chemical bond. The chemical reaction may comprise acceptance by the first chemical reagent of an electron from the second chemical reagent. A molecular and/or oligomeric machine may be configured to act as chemical catalysts, molecular assemblers and/or destructors capable of synthesizing polymeric materials, and/or energy concentrators. A molecular and/or oligomeric machine may be configured to facilitate chemical reactions by bringing reagents close to each other, or by transporting energetic/charged reagents.

Other exemplary embodiments may include bistable oligomeric machines configured to respond to multiple different stimuli. A molecular and/or oligomeric machine may comprise an arrangement of molecules configured to perform a mechanical function in response to application of a first stimulus, wherein the mechanical function is selected from a group consisting of rotating, lifting, pressing, ratcheting, springing, and flexing, and a receptor associated with the arrangement of molecules, the receptor being configured to receive a second stimulus different from the first stimulus, and to deactivate the mechanical function in response to the second stimulus, despite a continued application of the first stimulus. The first and second stimuli may be independently selected so as to comprise any of a change in temperature and/or a compound configured to bind to the receptor and thereby deactivate the mechanical function. The molecular and/or oligomeric machine may be configured to be irreversibly inoperable when bound by an inactivating compound. The receptor may be configured to make the molecular and/or oligomeric machine more easily excreted from a mammalian body. The receptor may be configured such that when triggered the molecular and/or oligomeric machine becomes more polar.

Unless otherwise defined, all technical and scientific terms used herein have the same meaning as commonly understood by one of ordinary skill in the art. In case of conflict, the present document, including definitions, will control. Preferred methods and materials are described herein, although methods and materials similar or equivalent to those described herein can be used in practice or testing of embodiments of the present disclosure. The materials, methods, and examples disclosed herein are illustrative only and not intended to be limiting. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an,” and “the” include plural references unless the context clearly dictates otherwise. The present disclosure also contemplates other embodiments “comprising,” “consisting of,” and “consisting essentially of” the embodiments or elements presented herein, whether explicitly set forth or not. The conjunctive term “or” may include any and all combinations of one or more listed elements associated by the conjunctive term. For example, the phrase “an apparatus comprising a or b” may refer to an apparatus including a where b may be not present, an apparatus including b where a may be not present, or an apparatus where both a and b are present. The phrases “at least one of a, b, . . . , and n” or “at least one of a, b, . . . , n, or combinations thereof” are defined in the broadest sense to mean one or more elements selected from the group comprising a, b, . . . , and n, that is to say, any combination of one or more of the elements a, b, . . . , or n including any one element alone or in combination with one or more of the other elements, which may also include, in combination, additional elements not listed. The terms “first,” “second,” “third,” and the like, as used herein, do not denote any order, quantity, or importance, but rather are used to distinguish one element from another. The term “substantially,” as used herein, represents the inherent degree of uncertainty that may be attributed to any quantitative comparison, value, measurement, or other representation. The term “substantially” may be also utilized herein to represent the degree by which a quantitative representation may vary from a stated reference without resulting in a change in the basic function of the subject matter at issue. The term “at least one bending location or one hinge location” refers to at least one position of co-joinder between the at least two oligomeric modules that allow the at least two oligomeric modules to predictably flex relative to each other about the bending or hinge location.

What is claimed is:

1. An oligomeric machine component configured to exhibit conformational bistability, comprising:
a first oligomeric module having a first end and a second end;
a second oligomeric module having a first end and a second end,
wherein the first end of the first oligomeric module is joined to the first end of the second oligomeric module; and
wherein a relative orientation of the first oligomeric module and the second oligomeric module changes from a first orientation to a second orientation in response to an applied stimulus.

2. The oligomeric machine component according to claim 1, wherein the oligomeric machine component repeatedly fluctuates between the first orientation and second orientation in response to the applied stimulus.

3. The oligomeric machine component according to claim 1, wherein an applied stimulus is a first stimulus and the relative orientation of the first oligomeric module and the second oligomeric module changes from the second orientation to the first orientation upon application of an additional stimulus and/or cessation of the first stimulus.

4. The oligomeric machine component according to claim 1, wherein the applied stimulus is any one or more of a change in temperature, a set temperature, an electric field, a magnetic field, a change in pH, an applied force of at least 10 picoNewtons, a prescribed amount of energy, and/or a change in ionic strength.

5. The oligomeric machine component according to claim 1, further comprising at least one bending or hinge location at a position of co-joinder between the first and second oligomeric modules.

6. The oligomeric machine component according to claim 5, wherein the at least one bending or hinge location comprises a third oligomeric module having a flexibility substantially greater than the flexibility of each of the first and second oligomeric modules.

7. The oligomeric machine component according to claim 6, wherein the applied stimulus causes the third oligomeric module to flex while the first and second oligomeric modules remains substantially un-flexed.

8. The oligomeric machine component according to claim 1, further comprising at least one extender formed of a material different from the first and second oligomeric modules and having a flexibility less than the flexibility of the oligomeric modules.

9. The oligomeric machine component according to claim 1, wherein each oligomeric segment has a length from 0.5 nm up to 15 nm.

10. The oligomeric machine component according to claim 1, wherein at least one oligomeric module comprises at least 15 repeating units of stereo-regular or stereo-irregular Poly(N-isopropylacrylamide).

11. The oligomeric machine component according to claim 1, wherein at least one oligomeric module comprises at least 15 repeating units of stereo-regular or stereo-irregular Poly(N-isopropylmethacrylamide).

12. The oligomeric machine component according to claim 1, wherein the first oligomeric module comprises at least 5 repeating units of Poly(N-isopropylacrylamide) in the stereo-regular or the stereo-irregular form, the second oligomeric module does not comprise Poly(N-isopropylacrylamide) and has a persistence length of at least 0.5 nm, and at least a one-third portion of the first oligomeric module does not flex more than 50% along a length of the at least one-third portion.

13. The oligomeric machine component according to claim 1, wherein at least one of the first or second oligomeric modules comprises an isotactic block.

14. The oligomeric machine component according to claim 1, wherein at least one of the first or second oligomeric modules comprises an atactic block.

15. The oligomeric machine component according to claim 1, wherein at least one of the first or second oligomeric modules comprises a syndiotactic block.

16. The oligomeric machine component according to claim 1, wherein the first and second oligomeric modules are selected and joined such that the oligomeric machine component is configured to have a conformational transitional temperature within 250 K to 400 K.

17. The oligomeric machine component according to claim 1, wherein the first and second oligomeric modules comprise a common monomeric unit.

18. The oligomeric machine component according to claim 1, further comprising a rigid molecular structure such as a nanotube or DNA.

19. The oligomeric machine component according to claim 1, wherein the joined first and second oligomeric modules comprises an isotactic Poly(N-isopropylacrylamide) block and an atactic Poly(N-isopropylacrylamide) block.

20. The oligomeric machine component according to claim 1, wherein the joined first and second oligomeric modules comprises a Poly(N-isopropylmethacrylamide) block between two Poly(2-Isopropyl-N-methylacrylamide) blocks.

21. An oligomeric machine, comprising:
a synthetic material including a first oligomeric module and a second oligomeric module joined to the first oligomeric module to form an oligomeric chain;
at least one bending or hinge location at a position of co-joinder between the first oligomeric module and the second oligomeric module, the bending or hinge location permitting relative flexure between the first oligomeric module and the second oligomeric module;
at least one electric generating element;
a substrate configured relative to the at least one electric generating element and the oligomeric chain such that the relative flexure between the first oligomeric module and the second oligomeric module results in mechanical interaction between at least the second oligomeric module of the oligomeric chain and the at least one electric generating element; and
wherein the oligomeric chain is formed such that in response to a stimulus, the relative flexure occurs between the first oligomeric module and the second oligomeric module in a manner causing the mechanical interaction between the second oligomeric module and the electric generating element, and wherein the mechanical interaction produces a change in electrical voltage associated with the at least one electric generating element.

22. The oligomeric machine according to claim 21, further comprising at least one light-absorbing element attached to the oligomeric chain at the at least one bending or hinge location wherein the oligomeric chain with light-absorbing element is formed such that in response to a prescribed amount of light energy applied to the light-absorbing element, the relative flexure occurs between the first oligomeric module and the second oligomeric module in a manner causing the mechanical interaction between the second oligomeric module and the at least one electric generating element to produce a change in electrical voltage associated with the at least one electric generating element.

23. The oligomeric machine according to claim 21, further comprising at least one chemically specific site disposed at the at least one bending or hinge location of the oligomeric chain for selective binding of a detectable molecule wherein the oligomeric chain with the chemically specific site is formed such that in response to binding of a detectible molecule, the relative flexure occurs between the first oligomeric module and the second oligomeric module in a manner causing the mechanical interaction between the second oligomeric module and the at least one electric generating element to produce a change in electrical voltage associated with the at least one electric generating element.

24. The oligomeric machine according to claim 21, wherein the electric generating element includes at least one of a piezoelectric element, nano-particle, nano-wire, or nano-layer.

25. The oligomeric machine according to claim 21, wherein the oligomeric chain comprises a Poly(N-isopropylmethacrylamide) block between two Poly(2-Isopropyl-N-methylacrylamide) blocks.

26. An oligomeric drug delivery machine, comprising:
a first oligomeric module;
a second oligomeric module connected to the first oligomeric module at a bend or hinge location to form an oligomeric chain;
a therapeutic agent captured between the first oligomeric module and the second oligomeric module;
wherein the oligomeric chain is configured such that upon application of energy thereto, relative movement occurs between the first oligomeric module and the second oligomeric module such that the captured therapeutic agent is released.

27. The oligomeric drug delivery machine according to claim 26, further comprising a targeting agent configured to facilitate delivery of the therapeutic agent in a targeted manner to at least one organ in a mammalian body.

28. The oligomeric drug delivery machine according to claim 27, wherein the targeting agent comprises an antibody.

29. The oligomeric drug delivery machine according to claim 26, wherein the therapeutic agent is captured between the first oligomeric module and the second oligomeric module by a non-covalent bond.

30. The oligomeric drug delivery machine according to claim 26, wherein the therapeutic agent is captured between the first oligomeric module and the second oligomeric module by a chemical bond.

31. The oligomeric drug delivery machine according to claim 30, wherein the chemical bond is a hydrolysable bond, and wherein in response to the relative flexure occurring between the first oligomeric module and the second oligomeric module, the hydrolysable bond is exposed to a solvent, which is configured to break the hydrolysable bond and release the captured therapeutic agent.

32. The oligomeric drug delivery machine according to claim 26, wherein the therapeutic agent is encapsulated within an encapsulation structure arranged for engagement by the oligomeric chain such that application of energy to the oligomeric chain induces the relative flexure between the first oligomeric module and the second oligomeric module in a manner causing rupture of the encapsulation structure and release of the therapeutic agent.

33. The oligomeric drug delivery machine according to claim 26, wherein the oligomeric chain comprises a Poly(N-isopropylmethacrylamide) block between two Poly(2-Isopropyl-N-methylacrylamide) blocks.

34. An oligomeric machine, comprising:
an arrangement of molecules configured for introduction into a mammalian body, the molecules being arranged and selected such that when exposed to a prescribed temperature, the arrangement performs at least one mechanical function selected from a group consisting of vibrations, extending, rotating, lifting, pressing, ratcheting, springing, and flexing;
wherein the prescribed temperature is a normal mammalian body temperature and is below a temperature causing necrosis to mammalian cells, such that upon introduction of the molecular arrangement to the mammalian body, the molecular arrangement does not perform the mechanical function until the arrangement of molecules is exposed to a temperature at least equal to the prescribed temperature.

35. The oligomeric machine according to claim 34, further comprising a sensitizer configured to increase a local temperature.

36. The oligomeric machine according to claim 35, wherein the sensitizer is chemically attached to the arrangement of molecules.

37. The oligomeric machine according to claim 36, wherein the sensitizer includes at least one of a nanoparticle and an organic dye.

38. The oligomeric machine according to claim 35, wherein the sensitizer is configured to absorb light in the infra-red spectrum.

39. The oligomeric machine according to claim 34, wherein the oligomeric machine is chemically attached to an antibody.

40. The oligomeric machine according to claim 34, wherein the oligomeric machine is configured as a molecular linear actuator.

41. The oligomeric machine according to claim 34, wherein the oligomeric machine comprises at least one imaging agent that activates after the oligomeric machine performs a mechanical function.

42. The oligomeric machine according to claim 34, wherein the oligomeric machine is configured as a molecular tweezer, wherein the at least one mechanical function contracts at least two distal ends of the oligomeric machine to grasp an object between the distal ends of the oligomeric machine.

43. The oligomeric machine according to claim 34, wherein the oligomeric machine is chemically attached to a payload, and the oligomeric machine is configured as a molecular shuttle to move the payload.

44. The oligomeric machine according to claim 34, wherein the arrangement of molecules comprises a Poly(N-isopropylmethacrylamide) block between two Poly(2-Isopropyl-N-methylacrylamide) blocks.

45. An oligomeric machine, comprising:
a synthetic material including a first oligomeric module and a second oligomeric module joined to the first oligomeric module to form an oligomeric chain;
at least one bend or hinge location at a position of co-joinder between the first oligomeric module and the second oligomeric module, the bend or hinge location permitting relative flexure between the first oligomeric module and the second oligomeric module; and
a first chemical reagent attached to the first oligomeric module;
a second chemical reagent attached to the second oligomeric module; and
wherein the oligomeric chain is formed such that in response to a prescribed amount of energy applied thereto, the first chemical reagent and the second chemical reagent are caused to be drawn into contact with each other and to undergo a chemical reaction.

46. The oligomeric machine according to claim 45, wherein the chemical reaction includes a chemical bond.

47. The oligomeric machine according to claim 45, wherein the chemical reaction includes acceptance by the first chemical reagent of an electron from the second chemical reagent.

48. The oligomeric machine according to claim 45, wherein the oligomeric chain comprises a Poly(N-isopropylmethacrylamide) block between two Poly(2-Isopropyl-N-methylacrylamide) blocks.

49. An oligomeric machine, comprising:
a synthetic material including a first oligomeric module and a second oligomeric module joined to the first oligomeric module to form an oligomeric chain;
at least one bending or hinge location at a position of co-joinder between the first oligomeric module and the second oligomeric module, the bending or hinge location permitting relative flexure between the first oligomeric module and the second oligomeric module;
at least one piston element;
a substrate configured relative to the at least one piston element and the oligomeric chain such that the relative flexure between the first oligomeric module and the second oligomeric module results in mechanical interaction between at least the second oligomeric module of the oligomeric chain and the at least one piston element; and
wherein the oligomeric chain is formed such that in response to a prescribed amount of energy applied thereto, the relative flexure occurs between the first oligomeric module and the second oligomeric module in a manner causing the mechanical interaction between the second oligomeric module and the piston element, and wherein the mechanical interaction produces a mechanical force.

50. The oligomeric machine according to claim 49, wherein the piston element is at least one of a graphene nanotube, a nano-wire, or a DNA fragment.

51. The oligomeric machine according to claim 49, wherein the oligomeric chain comprises a Poly(N-isopropylmethacrylamide) block between two Poly(2-Isopropyl-N-methylacrylamide) blocks.
